(19) United States
(12) Patent Application Publication
(10) Pub. No.: US 2003/0157166 A1
(43) Pub. Date: Aug. 21, 2003

(54) Controlled release sulfonylurea formulation

(76) Inventors: Chih Ming Chen, Davie, FL (US); Joseph Chou, Manassas, VA (US); David Wong, Milpitas, CA (US)

Correspondence Address: Davidson, Davidson & Kappel, LLC, 14th Floor, 485 Seventh Avenue, New York, NY, 10018, US

(21) Appl. No.: 10/101,438

(22) Filed: Mar. 18, 2002

Related U.S. Application Data
(60) Provisional application No. 60/276,447, filed on Mar. 16, 2001.

(51) Int. Cl. A61K 9/22; A61K 31/426

(52) U.S. Cl. 424/468; 514/369

(57) ABSTRACT

Disclosed in a controlled release sulfonylurea formulation. In certain embodiments, the invention comprises:

(a) a core comprising:

(i) a sulfonylurea or a pharmaceutically acceptable salt thereof;

(ii) a pharmaceutically acceptable polymer;

(b) a membrane surrounding the core which is permeable to the sulfonylurea and gastrointestinal fluid, wherein said dosage form provides a mean time to maximum plasma concentration (Tₘₐₓ) of said sulfonylurea.

This application claims priority from U.S. Provisional Application No. 60/276,447 filed Mar. 16, 2001, the disclosure of which is hereby incorporated by reference in its entirety.

### FIELD OF THE INVENTION

The invention is directed to controlled release formulations containing a sulfonylurea or derivative compounds which are suitable for administration to a patient in need of treatment related thereto. More specifically, the present invention relates to an oral dosage form comprising a sulfonylurea such as glipizide or a pharmaceutically acceptable salt thereof which is described in United States Pharmacopeia, National Formulary, Glipizide, (1995) pp. 707-708, which is incorporated herein by reference.

### BACKGROUND OF THE INVENTION

It will be appreciated by those skilled in the art that controlled or sustained release compositions employing a sulfonylurea or derivative compound would be particularly useful in the treatment of diabetes mellitus or for the treatment of those in clinical need of blood-glucose lowering therapy.

Diabetes mellitus is a metabolic disorder characterized by hyperglycemia, insulin resistance, and is often associated with other disorders such as obesity, hypertension, hyperlipidemia, as well as complications such as cardiovascular disease, retinopathy, neuropathy, and nephropathy. The disease is progressive in nature, and can often be controlled initially by diet alone, but generally requires treatment with drugs such as sulfonylureas (e.g., glipizide).

Glipizide is an oral hypoglycemic drug used in the management of non-insulin-dependent diabetes mellitus (NIDDM). Glipizide is useful therapeutically as an oral hypoglycemic drug because it stimulates insulin secretion from the pancreas (an effect dependent upon beta cells in the pancreatic islets), and because it exhibits extrapancreatic action such as the ability to increase insulin sensitivity and decrease hepatic glucose production. Glipizide is known chemically as 1-cyclohexylamino-3-[[p-[2-5-methylpyrazinecarboxamido)ethyl]phenyl]sulfonyl]urea. Peak plasma concentrations of glipizide occur 1 to 3 hours after a single oral dose, and the half life of elimination ranges from 2-4 hours whether given intravenously or orally.

Glipizide is commercially available, for example, under the tradename GLUCOTROL® tablets by Pfizer Inc. Each GLUCOTROL® tablet contains 2.5, 5 or 10 mg of glipizide. There is no fixed regimen for GLUCOTROL®, and the dose is typically individualized based on tolerance and effectiveness. Typically the recommended starting is 5 mg (2.5 mg in geriatric or patients with liver disease), with dosage adjustments based on blood glucose response. Glipizide is described in the 54^{th} Edition of the Physicians' Desk Reference, copyright 2000, p. 2345 as a whitish, odorless powder with a pKa of 5.9, and is insoluble in both water and alcohol. These physical and chemical properties of glipizide do not easily lend the drug to formulation into a dosage form which provides glipizide at a controlled and known rate per unit time to produce the intended therapy.

Extended release sulfonylurea formulations with improved dissolution properties, are therefore a desirable addition to the medical treatment of diabetes, including type II diabetes. Of these drugs, efforts to provide controlled release have focused on glipizide. For example, Glucotrol XL® Extended Release Tablet(Pfizer Inc; 2.5, 5 and 10 mg unit doses) is an extended release glipizide formulation available as osmotic based dosage forms. Specifically, Glucotrol XL® is prepared as an osmotically active drug core surrounded by a semipermeable membrane. The core itself is divided into two layers: an “active” layer containing the drug, and a “push” layer containing pharmacologically inert (but osmotically active) components. The membrane surrounding the tablet is permeable to water but not to drug or osmotic excipients. As water from the gastrointestinal tract enters the osmotically active material, the tablet pressure increases in the osmotic layer and “pushes” against the drug layer, resulting in the release of drug through a small laser-drilled orifice in the membrane on the drug side of the tablet.

In view of the above discussion, it will be appreciated by those skilled in the art, that a need exists for dosage forms that can deliver a sulfonylurea in a controlled release to a patient in clinical need of blood-glucose lowering therapy, and methods of use thereof.

### OBJECTS AND SUMMARY OF THE INVENTION

It is an object of the present invention to provide a controlled or sustained release of a hypoglycemic drug which provides effective control of blood glucose levels in humans.

It is a further object of the present invention to provide a method of treating human patients with non-insulin-dependent diabetes mellitus (NIDDM) on a once-a-day or twice a day basis with a hypoglycemic drug which provides effective control of blood glucose levels in humans.

It is a further object of the present invention to provide formulations for treating human patients with non-insulin-dependent diabetes mellitus (NIDDM) which provides advantages over the state-of-the-art, and which may be administered on a once-a-day or twice-a-day basis by itself or together with other antidiabetic agents, and methods thereof.

It is also a further object of the present invention to provide a controlled or sustained release formulation of a hypoglycemic drug that can provide therapeutic levels of the hypoglycemic drug to an animal or human in need of such treatment over a twelve hour to twenty-four hour period.

It is an additional object of other embodiments of the present invention to provide a controlled or sustained release formulation for a hypoglycemic drug that obtains peak plasma levels at from about 4 to about 16 hours and preferably at from about 6 hours to about 12 hours after administration under various conditions.

In accordance with the above-mentioned objects and others, the present invention in certain embodiments is directed to a controlled release solid oral dosage form comprising a sulfonylurea that is suitable for providing once-a-day administration of the drug, wherein the dosage form provides a mean time to maximum plasma concentration (Tₘₐₓ) of the drug at from about 4 to about 16 hours and preferably at from about 6 hours to about 12 hours after administration. The dosage form comprises the drug, a pharmaceutically acceptable polymer, and a membrane. In certain preferred embodiments, the dosage form comprises a tablet.

In preferred embodiments, the controlled release solid oral dosage form of the present invention is a tablet comprising:

(a) a core comprising:

(a) a sulfonylurea (e.g., glipizide);

(b) a pharmaceutically acceptable polymer;

(c) optionally a binding agent; and

(d) optionally an absorption enhancer;

(b) a permeable membrane coating surrounding the core; and

(c) optionally at least one passageway in the permeable membrane.

When the drug is glipizide or a pharmaceutically acceptable salt thereof and is administered on a once-a-day basis, the daily dose may vary, e.g., from about 1 mg to about 60 mg, from about 2 mg to about 40 mg, or from about 2.5 mg to about 10 mg, depending on the clinical needs of the patient. Such daily dose may be contained in one controlled-release dosage form of the invention, or may be contained in more than one such dosage form. For example, a controlled-release glipizide dosage form may be formulated to contain 2.5, 5 or 10 mg of the drug, and a combination of these dosage forms may be administered together to provide a desired once-a-day glipizide dose.

In preferred embodiments, the controlled release solid oral dosage form of the present invention provides a mean time to maximum plasma concentration of the drug (e.g., glipizide) from about 6 hours to about 12 hours after administration.

In certain embodiments, the controlled release oral dosage form of the present invention comprises a membrane coated core of a sulfonylurea and a polymer, with a passageway disposed through the membrane, wherein the release rate of the sulfonylurea and the amount of polymer have an inverse relationship.

In certain embodiments the controlled release oral dosage form of the present invention comprises a membrane coated core of a sulfonylurea and a polymer, with a passageway disposed through the membrane, wherein the membrane cracks within 1-5 hours after introduction to an aqueous medium to expose the core and increase the release rate.

The present invention is also directed to a method of lowering blood glucose levels in human patients needing treatment for non-insulin-dependent diabetes mellitus (NIDDM), comprising orally administering to human patients on a once-a-day basis a dose of a drug comprising a sulfonylurea (e.g., glipizide or a pharmaceutically acceptable salt thereof), the glipizide being contained in at least one solid oral controlled release dosage form of the present invention. When the drug is glipizide, the daily dose of the drug may be from about 1 mg to about 60 mg, from about 2 mg to about 40 mg, or from about 2.5 mg to about 10 mg, depending on the clinical needs of the patient. In certain embodiments, the method of treatment according to the present invention involves once-per-day glipizide monotherapy as an adjunct to diet to lower blood glucose in patients with NIDDM whose hyperglycemia may not be satisfactorily managed on diet alone. In certain other embodiments, the once-a-day glipizide therapy of the present invention may be used concomitantly with other diabetic agents as an adjunct to lower blood glucose in patients with NIDDM whose hyperglycemia may not be satisfactorily managed on diet alone.

The present invention is further directed to a method of controlling the serum glucose concentration in human patients with NIDDM, comprising administering to patients having NIDDM on a once-a-day basis, preferably prior to a morning meal, an effective dose of a sulfonylurea (e.g., glipizide) contained in at least one oral controlled release dosage form of the present invention.

The present invention further includes a controlled-release dosage form of a drug comprising sulfonylurea (e.g., glipizide) suitable for once-a-day administration to human patients with NIDDM, the dosage form comprising an effective amount of the drug to control blood glucose levels for up to about 24 hours and an effective amount of a controlled-release carrier to provide controlled release of the drug with a mean time to maximum plasma concentration (Tₘₐₓ) of the drug at from about 4 to about 16 hours and preferably at from about 6 hours to about 12 hours after administration. In preferred embodiments, the administration of the controlled-release dosage form occurs at fasted state, more preferably prior to breakfast time.

The drugs which may used in conjunction with the present invention include those drugs which are useful for the treatment of non-insulin-dependent diabetes mellitus (NIDDM), including but not limited to sulfonylureas such as glyburide (glibenclamide), glipizide and gliclazide or pharmaceutically acceptable salts thereof.

In alternate embodiments, the dosage form of the present invention can include a further anti-diabetic agent in addition to glipizide. For example, the dosage form can include metformin, buformin, another sulfonylurea such as glyburide (glibenclamid), chloropropamide, tolbutamide, acetohexamide and tolazamide, or pharmaceutically acceptable salts thereof.

The term “glipizide” as it is used herein means glipizide base or any pharmaceutically acceptable salt.

The term “dosage form” as it is used herein means at least one unit dosage form of the present invention (e.g. the daily dose of the hypoglycemic agent can be contained in 2 unit dosage forms of the present invention for single once-a-day administration).

The term “morning” as it is used herein with respect to the dosing of the controlled release formulations of the invention means that the controlled release formulation is orally administered early in the day after the patient has awakened from overnight sleep, generally between about 6 a.m. and 11 a.m. (regardless of whether breakfast is eaten at that time, unless so specified herein).

The term “therapeutically effective reduction” when used herein is meant to signify that blood glucose levels are reduced by approximately the same amount as an extended release reference standard (e.g., GLUCOTROL XL®), when the controlled release dosage form is orally administered to a human patient on a once-a-day basis.

The term “sustained release” and “controlled release” are used interchangeably in this application and are defined for purposes of the present invention as the release of the drug from the dosage form at such a rate that when a once-a-day dose of the drug is administered in the sustained release or controlled-release form, blood (e.g., plasma) concentrations (levels) of the drug are maintained within the therapeutic range but below toxic levels over a period of time from about 12 to about 24 hours. When the drug used in the present invention is glipizide the controlled release solid oral dosage form containing such drug is also referred to as “GLIPIZIDE XL” or “GLIPIZIDE ER”.

The term “Cₘₐₓ” is the highest plasma concentration of the drug attained within the dosing interval, i.e., about 24 hours.

The term “Tₘₐₓ” is the time period which elapses after administration of the dosage form at which the plasma concentration of the drug attains the highest plasma concentration of drug attained within the dosing interval (i.e., about 24 hours).

The term “AUC” as used herein, means area under the plasma concentration-time curve, as calculated by the trapezoidal rule over the complete 24-hour interval.

The term “single dose” means that the human patient has received a single dose of the drug formulation and the drug plasma concentration has not achieved steady state.

The term “multiple dose” means that the human patient has received at least two doses of the drug formulation in accordance with the dosing interval for that formulation (e.g., on a once-a-day basis). Patients who have received multiple doses of the controlled release formulations of the invention may or may not have attained steady state drug plasma levels, as the term multiple dose is defined herein.

The term “mean”, when preceding a pharmacokinetic value (e.g. mean Tₘₐₓ) represents the arithmetic mean value of the pharmacokinetic value taken from a population of patients unless otherwise specified.

The term “inverse relationship” with respect to release rate and amount of polymer means that as the amount of polymer increases, the release rate decreases, and vice versa.

When the membrane of the invention is disclosed as “surrounding” the core, it is understood that this does not preclude the existence of an intermediate layer between the membrane and the core, e.g. an intermediate film coating.

### BRIEF DESCRIPTION OF THE DRAWINGS

FIG. 1 is a graph which depicts the dissolution profile in a medium (pH 7.5 phosphate buffer) of a 10 mg controlled release glipizide formulation of the formulation described in Example 1 as tested according to the procedure described in United States Pharmacopeia XXIII, Apparatus 2 @ 50 rpm.

FIG. 2 is a graph which depicts the dissolution profile in a medium (pH 7.5 potassium phosphate buffer) of a 10 mg controlled release glipizide formulation of the formulation described in Examples 2 to 5 as tested according to the procedure described in United States Pharmacopeia XXIII, Apparatus 2 @ 50 rpm.

FIG. 3 is a graph which depicts the mean plasma glipizide concentration vs. time in the fasted state in an N=8 biostudy of Example 6.

FIG. 4 is a graph which depicts the mean plasma glipizide concentration vs. time in the fed state in an N=8 biostudy of Example 6.

FIG. 5 is a graph which depicts the mean plasma glipizide concentration vs. time in the fasted state in an N=9 biostudy of Examples 7 and 8.

FIG. 6 is a graph which depicts the mean plasma glipizide concentration vs. time in the fed state in an N=9 biostudy of Examples 7 and 8.

FIG. 7 is a graph which depicts the mean plasma glipizide concentration vs. time in the fasted state in an N=8 biostudy of Example 9.

FIG. 8 is a graph which depicts the mean plasma glipizide concentration vs. time in the fed state in an N=7 biostudy of Example 9.

FIG. 9 is a graph which depicts mean glipizide concentration vs. time in the fasting state in an N=27 biostudy of Example 9.

FIG. 10 is a graph which depicts mean glipizide concentration vs. time in the non-fasting state in an N=23 biostudy of Example 9.

FIG. 11 is a graph which depicts amount dissolved vs. time (pH 7.5) of Example 9.

FIG. 12 is a graph which depicts amount dissolved vs. time (pH 6.5) of Example 9.

### DETAILED DESCRIPTION OF THE INVENTION

The term hypoglycemic drugs as used in this specification refers to drugs that are useful in controlling or managing noninsulin-dependent diabetes mellitus (NIDDM). Preferably, the hypoglycemic drug is a sulfonylurea such as glipizide or a pharmaceutically acceptable salt thereof.

The method and dosage forms of the present invention provide the advantage of treating human patients with non-insulin-dependent diabetes mellitus (NIDDM) on a once-a-day basis with a hypoglycemic drug which provides effective control of blood glucose levels in human. In preferred embodiments, the present invention provides a controlled or sustained release formulation for glipizide that obtains peak plasma levels at from about 6 hours to about 12 hours.

In certain embodiments, the invention is directed to a dosage form comprising

(a) a core comprising:

(i) a sulfonylurea or a pharmaceutically acceptable salt thereof,

(ii) a pharmaceutically acceptable polymer;

(b) a membrane surrounding the core which is permeable to the sulfonylurea and gastrointestinal fluid; said dosage form being suitable for providing once-a-day oral administration of the sulfonylurea.

Other embodiments are directed to a controlled release oral dosage form comprising:

(a) a core comprising:

(i) an agent consisting essentially of a sulfonylurea or a pharmaceutically acceptable salt thereof,

(ii) a pharmaceutically acceptable polymer;

(b) a membrane surrounding the core which is permeable to the sulfonylurea and gastrointestinal fluid; said dosage form being suitable for providing once-a-day oral administration of the sulfonylurea.

Other embodiments are directed to a controlled release oral dosage form comprising:

(a) a core comprising:

(i) a sulfonylurea or a pharmaceutically acceptable salt thereof;

(ii) a pharmaceutically acceptable polymer;

(ii a disintigrant;

(b) a membrane surrounding the core which is permeable to the sulfonylurea and gastrointestinal fluid; said dosage form being suitable for providing once-a-day oral administration of the sulfonylurea.

The pharmaceutically acceptable polymer can include but is not limited to a hydroxyalkylcellulose (e.g., hydroxypropyl cellulose, hydroxypropylmethyl cellulose); polyalkylene oxide having a weight average molecular weight of 100,000 to 6,000,000 (e.g., poly(ethylene) oxide, poly(methylene oxide), poly(butylene oxide), and poly(hexylene oxide); poly(hydroxy alkyl methacrylate) having a molecular weight of from 25,000 to 5,000,000; poly(vinyl)alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a hydrogel forming copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anyhydride in the copolymer; Carbopol® acidic carboxy polymers having a molecular weight of 450,000 to 4,000,000; Cyanamer® polyacrylamides; cross-linked water swellable indenemaleic anhydride polymers; Goodrite® polyacrylic acid having a molecular weight of 80,000 to 200,000; starch graft copolymers; Aqua-Keeps® acrylate polymer polysaccharides composed of condensed glucose units such as diester cross-linked polyglucan and the like. Other polymers which form hydrogels are described in U.S. Pat. Nos. 3,865,108; 4,002,173 and 4,207,893 all of which are incorporated by reference. Mixtures of the aforementioned pharmaceutically acceptable polymers may also be used. In certain embodiments, the pharmaceutically acceptable polymer has a molecular weight of greater than 350,000. In other embodiments, the polymer has a molecular weight of greater than 350,000, but less than 4 million. In certain preferred embodiments, the pharmaceutically acceptable polymer has a molecular weight of greater than 750,000, but less than 4 million. In other preferred embodiments, the pharmaceutically acceptable polymer has a molecular weight of about 2 million.

In certain preferred embodiments, the pharmaceutically acceptable polymer is a polymer including ethylcellulose, cellulose acetate, cellulose propionate, cellulose acetate propionate, cellulose acetate butyrate, cellulose acetate phthalate, cellulose triacetate, polymethyl methacrylate, polyethyl methacrylate, polybutyl methacrylate, polyisobutyl methacrylate, polyhexyl methacrylate, poly isodecyl methacrylate, polylauryl methacrylate, polyphenyl methacrylate, polymethyl acrylate, polyisopropyl acrylate, polyisobutyl acrylate, polyoctadecyl acrylate, polyethylene, polypropylene, polyethylene oxide, polyethylene terephthalate, polyvinyl isobutyl ether, polyvinyl acetate, polyvinyl chloride, polyurethane or a mixture thereof.

Preferably, the polymer of the invention is water soluble, most preferably the water soluble polymer is polyethylene oxide.

In preferred embodiments, the sulfonylurea and the polymer are at least partially interdispersed. In certain embodiments, the sulfonylurea and the polymer comprise a homogenous mixture having a uniform dispersion.

The binding agent may be any conventionally known pharmaceutically acceptable binder such as polyvinyl pyrrolidone, hydroxypropyl cellulose, hydroxyethyl cellulose, ethylcellulose, polymethacrylate, waxes and the like. Mixtures of the aforementioned binding agents may also be used. The preferred binding agents is lactose. The binding agent comprises approximately about 0 to about 40% of the total weight of the core and preferably about 3% to about 15% of the total weight of the core.

The core may optionally comprise a disintegrant. Examples of some disintegrants for use in the present invention are croscarmellose sodium, crospovidone, alginic acid, sodium alginate, methacrylic acid DVB, cross-linked PVP, microcrystalline cellulose, polacrilin potassium, sodium starch glycolate, starch, pregelatinized starch and the like. Some preferable disintegrants are cross-linked polyvinylpyrrolidone (e.g. Kollidon CL), cross-linked sodium carboxymethylcellulose (e.g. Ac-Di-Sol), starch or starch derivatives such as sodium starch glycolate (e.g. Explotab®), or combinations with starch (e.g. Primojel), swellable ion-exchange resins, such as Amberlite IRP 88, formaldehyd-casein (e.g. Esma Spreng). Most preferably the disintegrant is sodium starch glycolate. The disintegrant comprises approximately 0 to 20% of the total weight of the core, and most preferably about 2% to about 15% of the total weight of the core.

The core may optionally comprise an absorption enhancer. The absorption enhancer can be any type of absorption enhancer commonly known in the art such as a fatty acid, a surfactant, a chelating agent, a bile salt or mixtures thereof. Examples of some preferred absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis (B-aminoethyl ether)-N,N,N,N-tetraacetic acid (EGTA). The absorption enhancer comprises approximately 0 to about 20% of the total weight of the core and most preferably about 2% to about 10% of the total weight of the core.

In certain embodiments, the core comprises a antihyperglycemic drug, a pharmaceutically acceptable polymer, a binder and an absorption enhancer. The core is preferably formed by wet granulating the core ingredients and compressing the granules with the addition of a lubricant into a tablet on a rotary press. The core may also be formed by dry granulating the core ingredients and compressing the granules with the addition of a lubricant into tablets or by direct compression. Alternatively, the ingredients can be blended followed by compression.

In addition to the above ingredients, the core may also contain suitable quantities of other materials, e.g. preservatives, diluents, lubricants, binders, granulating aids, colorants, flavorants and glidants that are conventional in the pharmaceutical art. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

Prior to coating the core with the membrane, an indentation can be made during compression by an indentation pin located on the press punch. Alternatively, the core can be indented after compression. By “indented” it is meant that there is a depression in the core of the present invention.

Prior to coating the core with the membrane, the core may be coated with a pharmaceutically acceptable film-coating, e.g., for stability purposes (e.g., coated with a moisture barrier), etc. For example, the core may be overcoated with a seal coating, preferably containing a pigment, a barrier agent, or any other pharmaceutically acceptable excipient such as hydroxypropylmethylcellulose and/or a polymethylmethacrylate. An example of a suitable material which may be used for such a hydrophilic coating is hydroxypropylmethylcellulose (e.g., Opadry®, commercially available from Colorcon, West Point, Pa.). In addition, sodium chloride may also be included in the seal coating. Any pharmaceutically acceptable manner known to those skilled in the art may be used to apply the coatings. For example, the coating may be applied using a coating pan or a fluidized bed. An organic, aqueous or a mixture of an organic and aqueous solvent is used for the hydrophobic polymer or enteric coating. Examples of suitable organic solvents are, e.g., isopropyl alcohol, ethanol, and the like, with or without water. Aqueous solvents are preferred for the overcoating procedures.

The core is coated with a membrane, preferably a polymeric membrane to form the controlled release tablet of the invention. The membrane is permeable to passage of external fluid such as water and biological fluids and to the passage of the hypoglycemic drug in the core. Materials that are useful in forming the membrane are cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, 4,036,228 and 4,11210 which are incorporated herein by reference. The most preferred membrane material is cellulose acetate comprising an acetyl content of 39.3 to 40.3%, commercially available from Eastman Fine Chemicals.

In an alternative embodiment, the membrane can be formed from the above-described polymers and a flux enhancing agent. The flux enhancing agent increases the volume of fluid imbibed into the core to enable the dosage form to dispense substantially all of the hypoglycemic drug through the permeable membrane, regardless of the presence of a passageway. The flux enhancing agent can be a water soluble material or an enteric material. Some examples of the preferred materials that are useful as flux enhancers are sodium chloride, potassium chloride, sucrose, sorbitol, mannitol, polyethylene glycol (PEG), propylene glycol, hydroxypropyl cellulose, hydroxypropyl methycellulose, hydroxyprophy methycellulose phthalate, cellulose acetate phthalate, polyvinyl alcohols, methacrylic acid copolymers and mixtures thereof. The preferred flux enhancer is PEG 400.

The flux enhancer may also be a drug that is water soluble or a drug that is soluble under intestinal conditions. If the flux enhancer is a drug, the present dosage form has the added advantage of providing an immediate release of the drug which is selected as the flux enhancer.

In certain embodiments, the flux enhancer is not sodium chloride, and sodium chloride is not present in the membrane.

The flux enhancing agent comprises approximately 0 to about 40% of the total weight of the coating, most preferably about 2% to about 20% of the total weight of the coating. The flux enhancing agent dissolves or leaches from the membrane to form paths in the membrane for the fluid to enter the core and dissolve the active ingredient.

In addition, the membrane can be formed with enteric material. As enteric coating material polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). Some preferred commercial enteric coating materials are EUDRAGIT® L 100-55, EUDRAGIT® L 30 D-55, EUDRAGIT® L 100, and EUDRAGIT® S 100.

The enteric coating material comprises approximately 0 to about 60% of the total weight of the coating, most preferably about 1% to about 40% of the total weight of the coating.

In alternate embodiments, the membrane may also be formed with commonly known excipients such as a plasticizer. Some commonly known plasticizers include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 15% of the plasticizer can be used based upon the total weight of the coating.

In certain preferred embodiments, the plasticizer is triacetin in a range from about 2% to about 15% based upon the total weight of the coating, preferably from about 5% to about 12% and most preferably from about 8% to about 12%. The presence of triacetin in the membrane also provides flux enhancing effect.

In certain embodiments the membrane may be further coated with a pharmaceutically acceptable film-coating or color coating. For example, the membrane may be overcoated with a film coating, preferably containing a pigment and a barrier agent, such as hydroxypropylmethylcellulose and/or a polymethylmethacrylate. An example of a suitable material which may be used for such a hydrophilic coating is hydroxypropylmethylcellulose (e.g., Opadry®, commercially available from Colorcon, West Point, Pa.). Any pharmaceutically acceptable manner known to those skilled in the art may be used to apply the coatings. In addition, the tablets may polished with candelilla wax powder.

In certain embodiments the passageway can be formed by drilling, including mechanical and laser drilling, through the membrane. Passageways and equipment for forming passageways are disclosed in U.S. Pat. Nos. 3,845,770, 3,916,899, 4,063,064, and 4,088,864.

In other embodiments, the passageway is formed by making an indentation onto the core prior to the membrane coating to form a weakened area of the membrane at the point of the indentation, to form a passageway in an environment of use.

As used herein the term passageway includes an aperture, orifice, bore, hole, weaken area or an erodible element such as a gelatin plug that erodes to form an osmotic passageway for the release of the antihyperglycemic drug from the dosage form. A detailed description of the passageway can be found in U.S. Pat. Nos. such as 3,845,770, 3,916,899, 4,034,758, 4,063,064, 4,077,407, 4,088,864, 4,783,337 and 5,071,607 (the disclosures of which are hereby incorporated by reference).

In certain embodiments, the membrane coating around the core is less than 10% of the total weight of the dosage form, preferably the membrane coating around the core will be from about 1% to about 7%, preferably from about 2% to about 5%, most preferably from about 3% to about 4% based on the total weight of the formulation.

The term “membrane” means a membrane that is permeable to both aqueous solutions or gastrointestinal fluids and to the active drug or pharmaceutical ingredient (e.g. the formulations of Examples 1-9). Thus, the membrane is permeable to drug and, in certain embodiments, drug is released through a hole or passageway in addition to the permeable membrane.

In an alternative embodiment, the dosage form of the present invention may also comprise an effective amount of the drug that is available for immediate release. The effective amount of antihyperglycemic drug for immediate release may be coated onto the membrane of the dosage form or it may be incorporated into the membrane.

In certain preferred embodiments, the release of the drug from the dosage form is controlled by both the pharmaceutically acceptable polymer of the core and the membrane surrounding the core.

In certain preferred embodiments, upon contact with enviromental fluids which permeate through the membrane, the polymer forms a hydrogel which controls the release of the drug from the dosage form.

In certain preferred embodiments, upon contact with environmental fluids which permeate through the membrane, the polymer forms a matrix which controls the release of the drug from the dosage form.

In certain preferred embodiments, the drug is released from the dosage form is controlled by both the pharmaceutically acceptable polymer of the core and the membrane surrounding the core and is suitable for once-a-day therapy.

In certain preferred embodiments, the controlled release solid oral dosage form exhibits the following dissolution profiles when tested in USP type 2 apparatus at 50 rpm in 900 ml of medium (pH 6.5 potassium phosphate buffer) at 37° C.: from 0 to about 25% or from 0 to about 20% of the drug (e.g., glipizide or a pharmaceutically acceptable salt thereof) released after 2 hours; from about 10% to about 55% or from about 10% to about 30% of the drug released after 6 hours; from about 40% to about 95% or from about 40% to about 80% of the drug released after 12 hours; and not less than about 70% of the drug released after 16 hours.

In certain preferred embodiments, the controlled release solid oral dosage form exhibits the following dissolution profiles when tested in USP type 2 apparatus at 50 rpm in 900 ml of medium (pH 7.5 potassium phosphate buffer) at 37° C.: from 0 to about 40% of the drug (e.g., glipizide or a pharmaceutically acceptable salt thereof) released after 2 hours; about 20% to about 90% of the drug released after 6 hours; not less than about 60% of the drug released after 12 hours; not less than about 70% of the drug released after 16 hours; and not less than 80% of the drug release after 20 hours.

In certain preferred embodiments, the controlled release solid oral dosage form of the present invention, after oral administration of a single dose to a human patient, provide a mean plasma concentrations of glipizide of from about 10 to about 150 ng/ml at 4 hours after administration, from about 75 to about 350 ng/ml at 8 hours after administration; from about 65 to about 275 ng/ml at 12 hours after administration and from about 25 to about 125 ng/ml at 24 hours after administration, based on a 10 mg dose of glipizide.

In certain preferred embodiments, the controlled release solid oral dosage form of the present invention, after oral administration of a single dose to a human patient, provides a mean plasma concentrations of glipizide of from about 15 to about 100 ng/ml at 4 hours after administration, from about 75 to about 150 ng/ml at 8 hours after administration; from about 100 to about 180 ng/ml at 12 hours after administration and from about 30 to about 100 ng/ml at 24 hours after administration, based on a 10 mg dose of glipizide.

In certain preferred embodiments, the controlled release solid oral dosage form of the present invention, after oral administration of a single dose to a human patient, provide a mean plasma concentrations of glipizide of from about 5 to about 75 ng/ml at 4 hours after administration, from about 35 to about 175 ng/ml at 8 hours after administration; from about 30 to about 135 ng/ml at 12 hours after administration and from about 10 to about 65 ng/ml at 24 hours after administration, based on a 5 mg dose of glipizide.

In certain preferred embodiments, the controlled release solid oral dosage form of the present invention, after oral administration of a single dose to a human patient, provides a mean plasma concentrations of glipizide of from about 7 to about 50 ng/ml at 4 hours after administration, from about 35 to about 75 ng/ml at 8 hours after administration; from about 50 to about 100 ng/ml at 12 hours after administration and from about 15 to about 50 ng/ml at 24 hours after administration, based on a 5 mg dose of glipizide.

In certain embodiments of the invention, the once-a-day administration of the glipizide dosage form provides a mean AUC₀₋₂₄ₕᵣ from about 80% to about 120%, preferably from about 90% to about 110% of the mean AUC₀₋₂₄ provided by once-a-day administration of an equivalent dose of a controlled release reference standard (GLUCOTROL X®).

The dosage form of the present invention can provide therapeutic levels of the hypoglycemic drug for twelve to twenty-four hour periods. In a preferred embodiment, the dosage form can be administered once-a-day, ideally prior to a meal, and provides therapeutic levels of the drug throughout the day with peak plasma levels being obtained between about 6-12 hours after administration.

In the preparation of the tablets of the invention, various conventional well known solvents may be used to prepare the granules and apply the external coating to the tablets of the invention. In addition, various diluents, excipients, lubricants, dyes, pigments, dispersants, etc. which are disclosed in Remington's Pharmaceutical Sciences, 1995 Edition may be used to optimize the formulations of the invention.

### DESCRIPTION OF CERTAIN PREFERRED EMBODIMENTS

The following examples illustrate various aspects of the present invention. They are not to be construed to limit the claims in any manner whatsoever.

### EXAMPLE 1

A controlled release tablet containing 10 mg of glipizide and having the following formula was prepared as follows:

| | Percentage of |
|---|---|
| I. Core | tablet |
| Glipizide | 2.98% |
| Disintegrant (Explotab ®) | 8.93% |
| Anhydrous Lactose | 54.63% |
| Polyox N 60 K (MW = 2,000,000) | 17.87% |
| Magnesium Stearate | 0.89% |
| Colloidal Silicon Dioxide (e.g., Cab-O-Sil) | 0.45% |
| Glyceryl Monostearate | 3.57% |
| Butylated Hydroxytoluene (BHT) | 0.03% |

The glipizide and other ingredients comprising the core are blended and pressed into a solid layered core tablet. After blending, the granules are compressed on a rotary press fitted with 11/32″ round standard concave punches (plain lower punch, upper punch with an approximately 1 mm indentation pin).

| | Percentage of |
|---|---|
| II. Seal Coating | tablet |
| Opadry Clear | 3.90% |
| Sodium Chloride | 1.30% |

The core tablet is seal coated with an Opadry material and sodium chloride or other suitable water-soluble material by first dissolving the Opadry material, preferably Opadry Clear, in purified water with sodium chloride. The Opadry solution is then sprayed onto the core tablet using a pan coater under the following conditions: exhaust air temperature of 38-42° C.; atomization pressure of 28-40 psi; and spray rate of 10-15 ml/min (atomization pressure and spray rate vary depending on the equipment used). The core tablet is coated with the sealing solution until a theoretical coating level of approximately 5% is obtained.

| | Percentage of |
|---|---|
| III. Membrane | tablet |
| Cellulose Acetate | 1.23% |
| Eudragit S100 | 0.62% |
| Triacetin | 0.23% |
| PEG 400 | 0.35% |

The cellulose acetate is dissolved in acetone while stirring with a homogenizer. The Eudragit S100, polyethylene glycol 400 and triacetin are added to the cellulose acetate solution and stirred until a clear solution is obtained. The clear coating solution is then sprayed onto the seal coated tablets in a fluidized bed coater employing the following conditions: product temperature of 16-22° C.; atomization pressure of approximately three bars; and spray rate of 120-150 ml/min (atomization pressure and spray rate vary depending on the equipment used). The sealed core tablet is coated until a theoretical coating level of approximately 2.5% is obtained.

| | Percentage of |
|---|---|
| IV. Color Coating | tablet |
| Opadry White | 3.00% |
| Candelilla Wax Powder | 0.03% |

The membrane coated tablet is color coated with an Opadry material, or other suitable water-soluble material by first dissolving the Opadry material, preferably Opadry White, in purified water. The solution is then sprayed onto the core tablet using a pan coater. After drying, candelilla wax powder is used as a polishing agent.

The resulting tablet is dissolution tested in a pH 7.5 medium according to the procedure described in United States Pharmacopeia XXIII, Apparatus 2 @ 50 rpm and found to have the following release profile set forth in Table 1:

TABLE 1

| Time (Hours) | % Dissolved (pH 6.5) | % Dissolved (pH 7.5) |
|---|---|---|
| 2 | 4 | 13 |
| 6 | 23 | 44 |
| 12 | 71 | 96 |
| 16 | 98 | 113 |

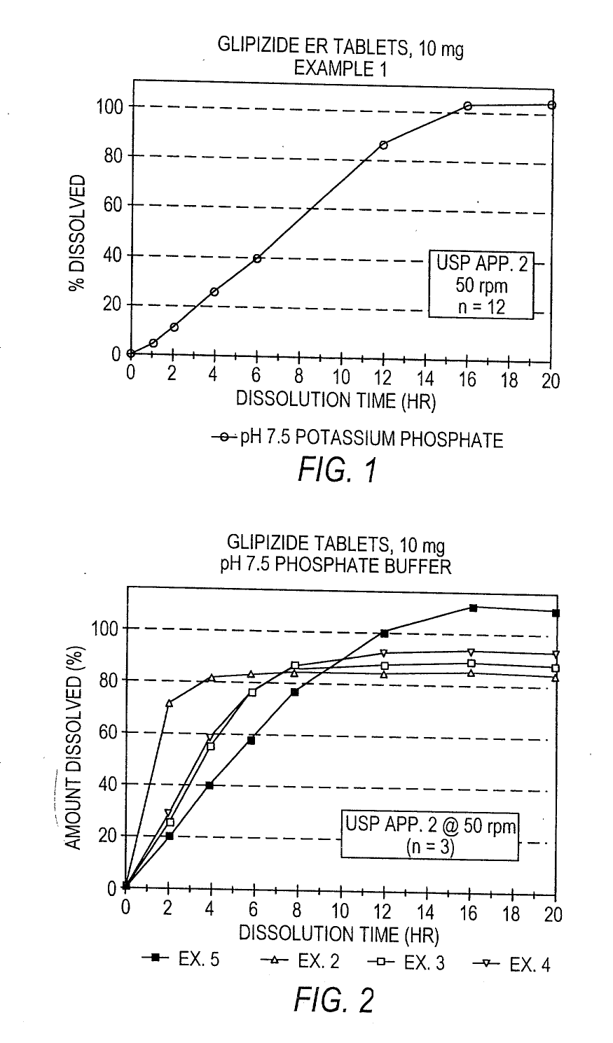

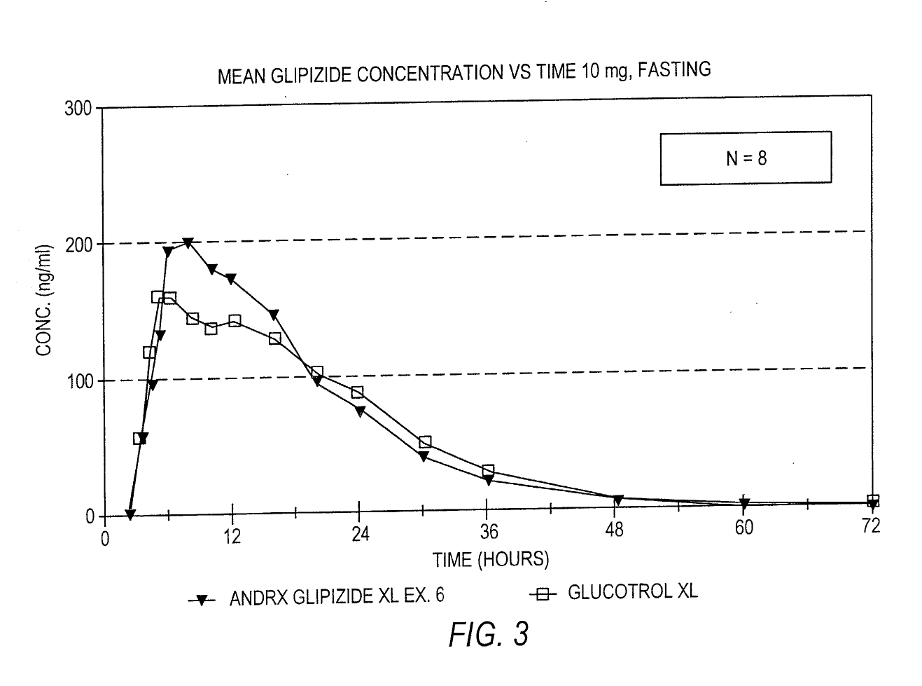

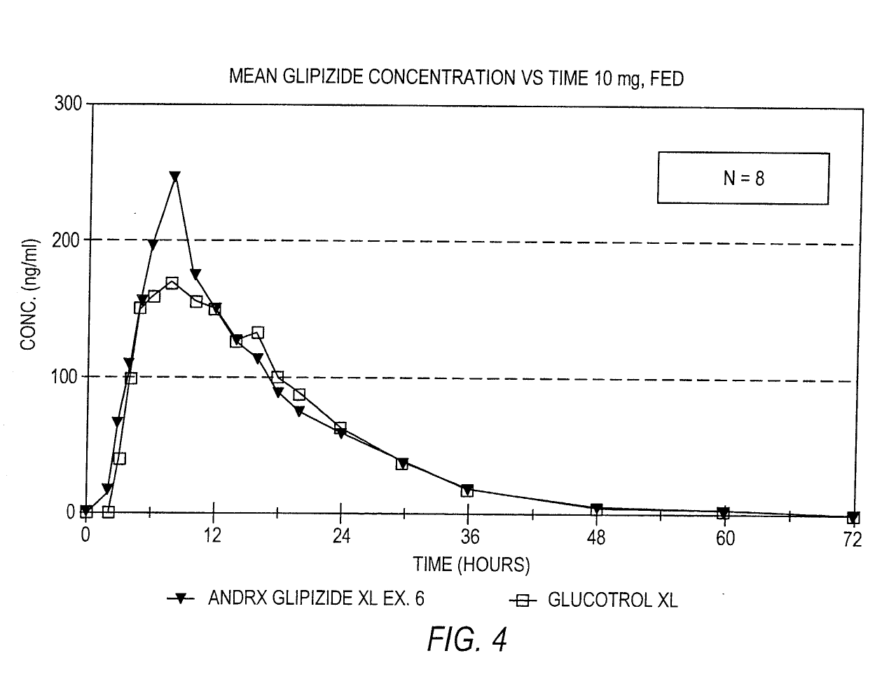

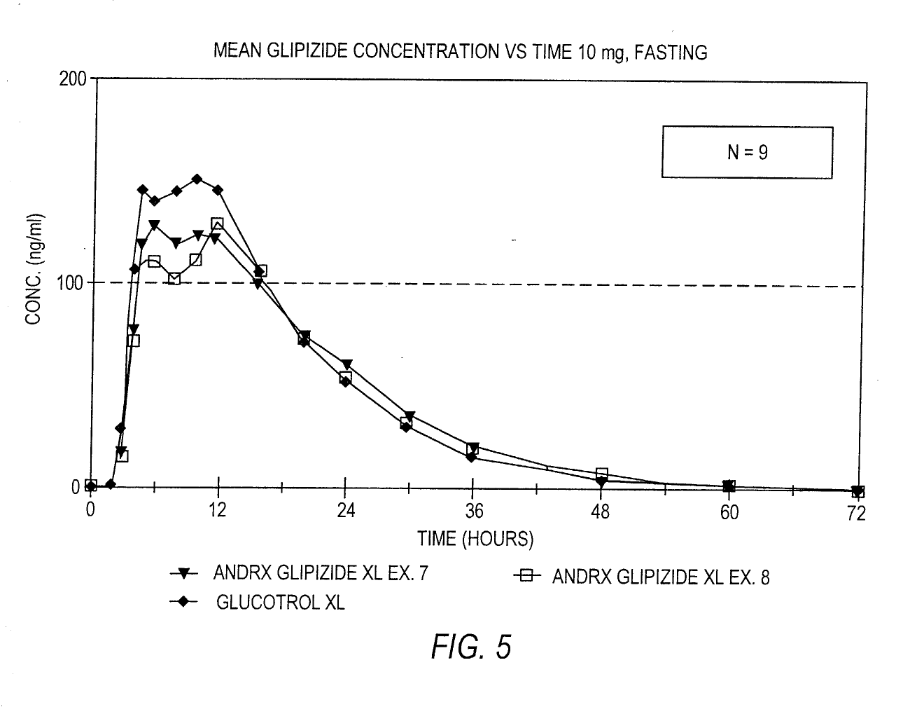

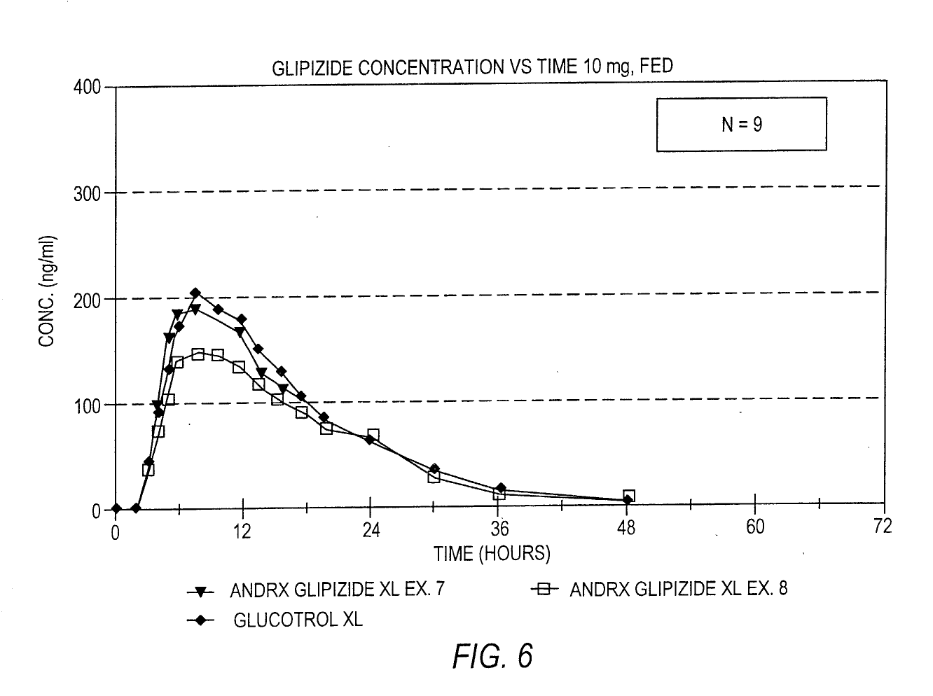

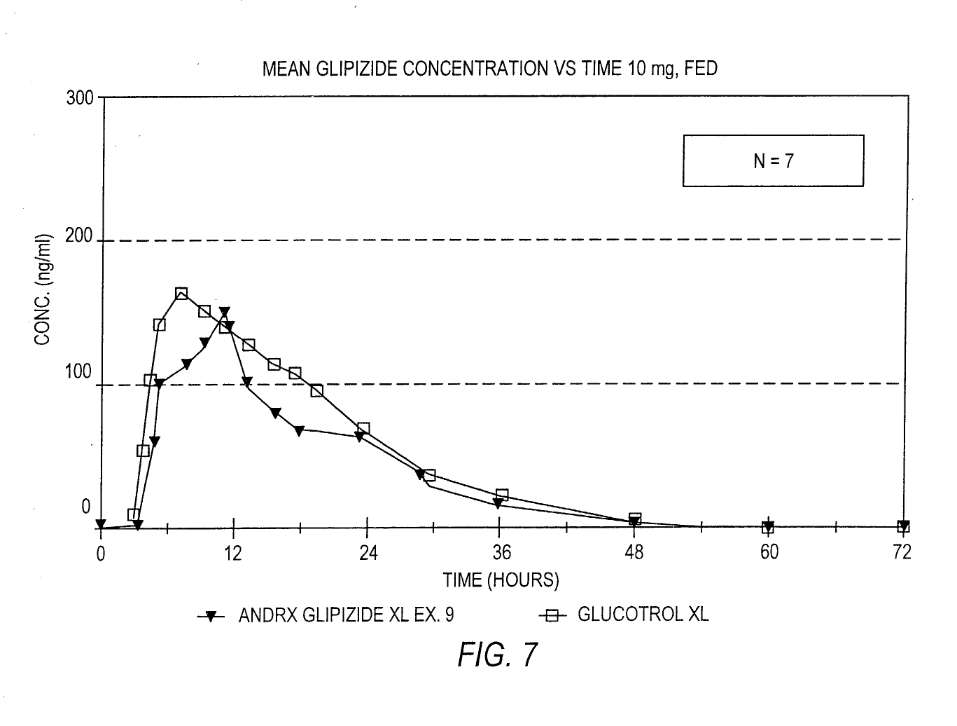

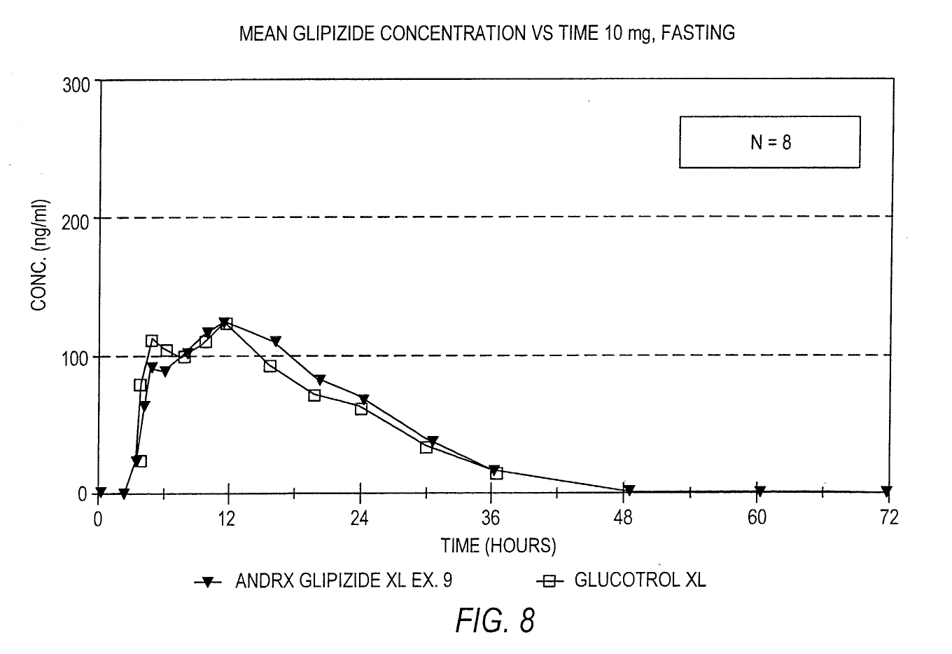

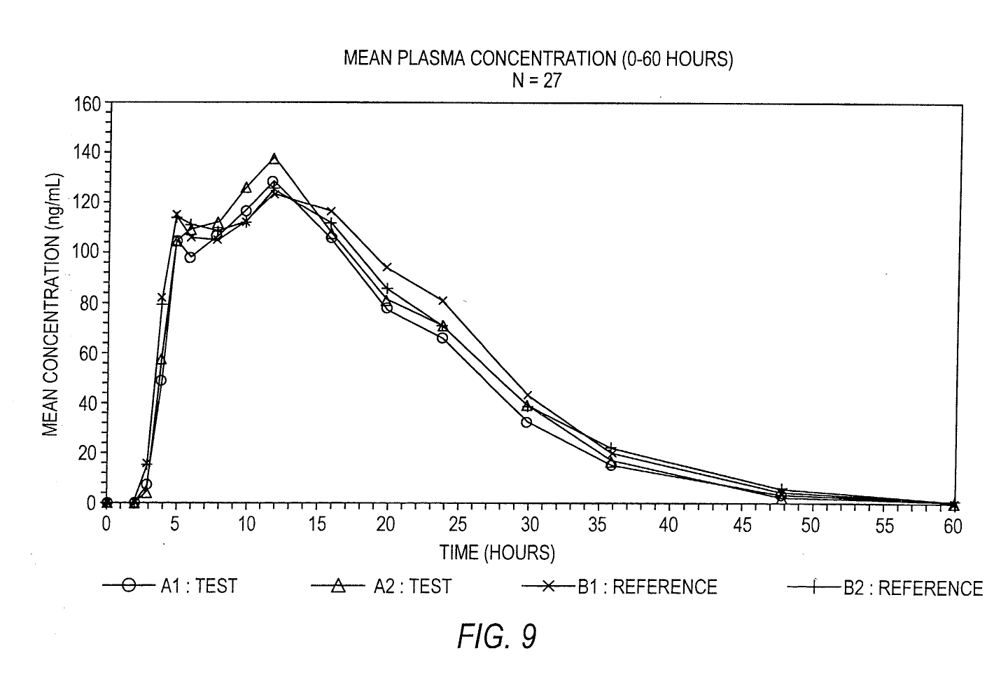

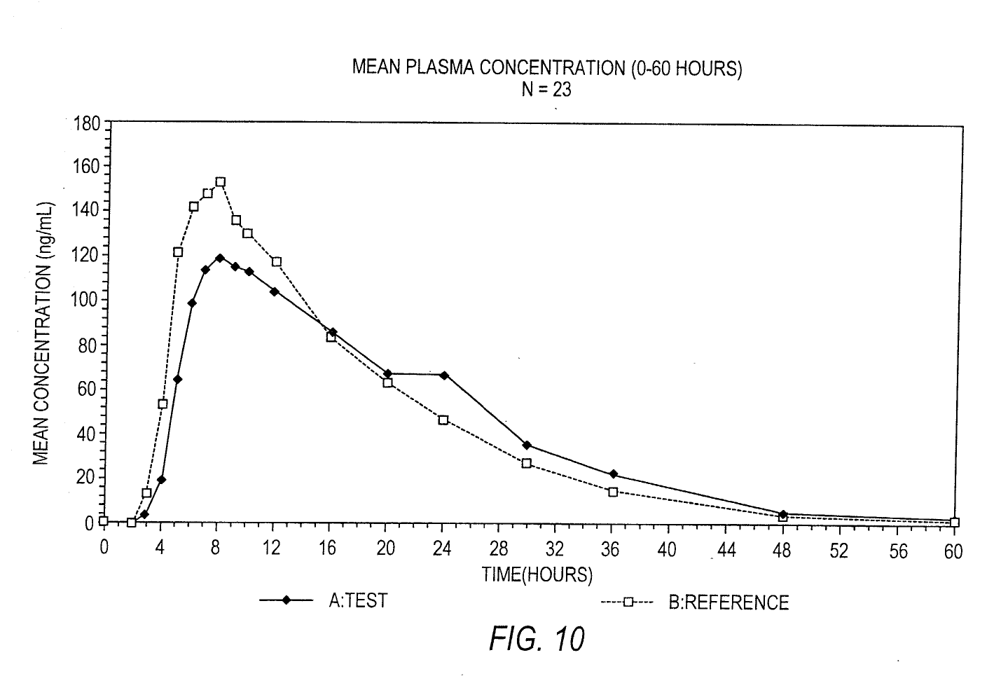

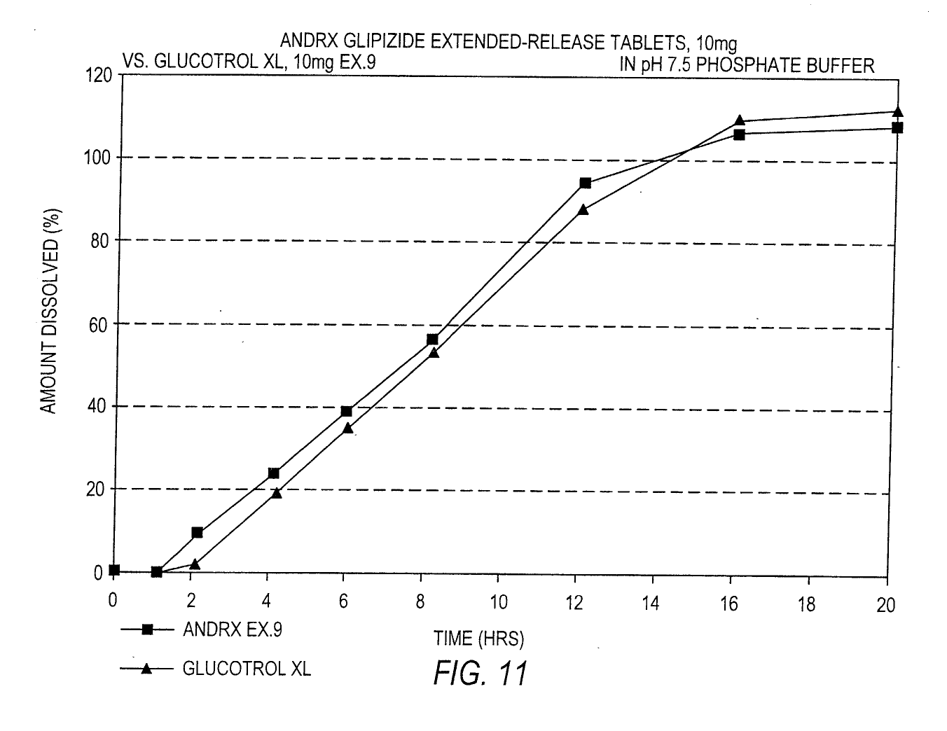

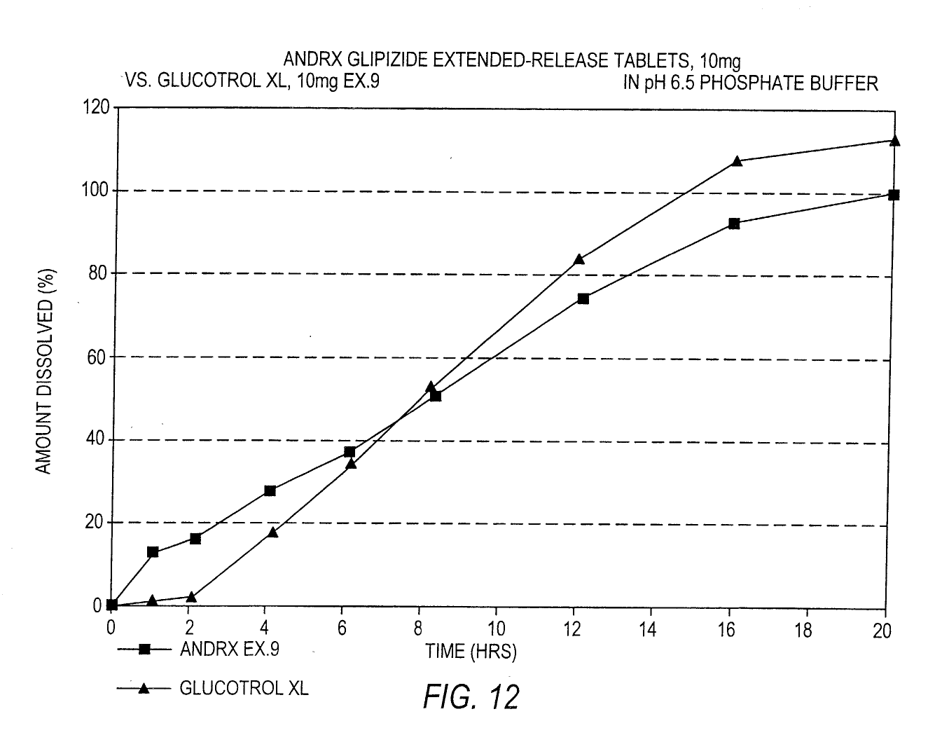

The in-vitro release profile in pH 7.5 of the sustained release product prepared in this Example is shown in FIG. 1.

Table 1A provides that individual plasma concentration (ng/ml) following fasting dosing with Example 1.

TABLE 1A

| | 0.00 | 1.00 | 2.00 | 3.00 | 4.00 | 5.00 | 6.00 | 8.00 | 10.00 | 12.00 |
|---|---|---|---|---|---|---|---|---|---|---|
| Subj | hr. | hr. | hr. | hr. | hr. | hr. | hr. | hr. | hr. | hr. |
| 1 | 0.00 | 0.00 | 0.00 | 0.00 | 88.50 | 211.42 | 339.44 | 354.66 | 219.20 | 144.14 |
| 2 | 0.00 | 0.00 | 27.94 | 87.27 | 98.32 | 220.20 | 226.69 | 145.65 | 151.50 | 101.62 |
| 3 | 0.00 | 0.00 | 0.00 | 64.70 | 260.88 | 371.93 | 500.24 | 345.16 | 229.93 | 187.64 |
| 4 | 0.00 | 0.00 | 0.00 | 104.75 | 141.40 | 237.93 | 356.31 | 385.38 | 280.72 | 234.10 |
| 5 | 0.00 | 0.00 | 28.46 | 93.38 | 144.96 | 176.25 | 265.37 | 535.10 | 428.70 | 316.95 |
| 6 | 0.00 | 0.00 | 0.00 | 0.00 | 39.78 | 104.72 | 68.63 | 354.60 | 279.65 | 196.43 |
| 7 | 0.00 | 0.00 | 0.00 | 0.00 | 31.63 | 77.17 | 80.52 | 111.89 | 112.13 | 502.30 |
| 8 | 0.00 | 0.00 | 0.00 | 0.00 | 52.52 | 207.95 | 428.01 | 333.40 | 302.18 | 195.97 |
| 9 | 0.00 | 0.00 | 0.00 | 0.00 | 0.00 | 0.00 | 0.00 | 76.66 | 274.14 | 297.29 |
| N | 9 | 9 | 9 | 9 | 9 | 9 | 9 | 9 | 9 | 9 |
| Mean | 0.00 | 0.00 | 6.27 | 38.90 | 95.33 | 178.62 | 251.69 | 293.61 | 253.12 | 241.83 |
| STDEV | 0.00 | 0.00 | 12.44 | 47.27 | 79.15 | 107.36 | 172.66 | 150.17 | 91.54 | 118.81 |
| %CV | | | 198.44 | 121.52 | 83.02 | 60.11 | 68.60 | 51.15 | 36.17 | 49.13 |
| MIN | 0.00 | 0.00 | 0.00 | 0.00 | 0.00 | 0.00 | 0.00 | 76.66 | 112.13 | 101.62 |
| MAX | 0.00 | 0.00 | 28.46 | 104.75 | 260.88 | 371.93 | 500.24 | 535.10 | 428.70 | 502.30 |

| | 14.00 | 16.00 | 18.00 | 20.00 | 24.00 | 30.00 | 36.00 | 48.00 | 60.00 | 72.00 |
|---|---|---|---|---|---|---|---|---|---|---|
| Subj | hr. | hr. | hr. | hr. | hr. | hr. | hr. | hr. | hr. | hr. |
| 1 | 94.66 | 65.44 | 51.54 | 38.13 | 25.03 | 0.00 | 0.00 | 0.00 | 0.00 | 0.00 |
| 2 | 100.24 | 50.00 | 34.75 | 0.00 | 0.00 | 0.00 | 0.00 | 0.00 | 0.00 | 0.00 |
| 3 | 137.95 | 99.76 | 75.30 | 93.72 | 39.67 | 0.00 | 0.00 | 0.00 | 0.00 | 0.00 |
| 4 | 166.67 | 130.31 | 101.07 | 78.87 | 59.21 | 34.82 | 0.00 | 0.00 | 0.00 | 0.00 |
| 5 | 233.63 | 158.51 | 104.91 | 70.90 | 43.16 | 0.00 | 0.00 | 0.00 | 0.00 | 0.00 |
| 6 | 135.99 | 93.78 | 69.66 | 51.29 | 31.06 | 0.00 | 0.00 | 0.00 | 0.00 | 0.00 |
| 7 | 357.16 | 289.25 | 208.76 | 160.60 | 103.19 | 49.31 | 39.34 | 0.00 | 0.00 | 0.00 |
| 8 | 163.92 | 257.31 | 226.68 | 161.69 | 87.28 | 42.53 | 0.00 | 0.00 | | 0.00 |
| 9 | 269.40 | 185.95 | 128.79 | 95.25 | 56.91 | 0.00 | 0.00 | 0.00 | 0.00 | 0.00 |
| N | 9 | 9 | 9 | 9 | 9 | 9 | 9 | 9 | 9 | |
| Mean | 184.40 | 147.81 | 111.27 | 83.38 | 49.50 | 14.07 | 4.37 | 0.00 | 0.00 | 0.00 |
| STDEV | 86.51 | 83.25 | 66.82 | 53.09 | 31.59 | 21.42 | 13.11 | 0.00 | 0.00 | 0.00 |
| %CV | 46.91 | 56.32 | 60.05 | 63.67 | 63.82 | 152.20 | 300.00 | | | |
| MIN | 94.66 | 50.00 | 34.75 | 0.00 | 0.00 | 0.00 | 0.00 | 0.00 | 0.00 | 0.00 |
| MAX | 357.16 | 289.25 | 226.68 | 161.69 | 103.19 | 49.31 | 39.34 | 0.00 | 0.00 | 0.00 |

### EXAMPLE 2

To demonstrate the effect of the Polyox on the release of the drug from the formulation, a controlled release tablet containing 10 mg of glipizide was prepared as in Example 1, having 0% Polyox N 60 K and 76.76% Anhydrous Lactose.

The resulting tablet is dissolution tested in a pH 7.5 medium according to the procedure described in United States Pharmacopeia XXIII, Apparatus 2 @ 50 rpm and found to have the following release profile set forth in Table 2:

TABLE 2

| | Mean % Dissolved (pH 7.5) |
|---|---|
| Time (Hours) | (n = 3) |
| 2 | 73 |
| 4 | 83 |
| 6 | 84 |
| 8 | 85 |
| 12 | 86 |
| 16 | 86 |
| 20 | 85 |

### EXAMPLE 3

A controlled release tablet containing 10 mg of glipizide was prepared as in Example 1, having 20% Polyox N 750 (MW=300,000).

The resulting tablet is dissolution tested in a pH 7.5 medium according to the procedure described in United States Pharmacopeia XXIII, Apparatus 2 @ 50 rpm and found to have the following release profile set forth in Table 3:

TABLE 3

| | Mean % Dissolved (pH 7.5) |
|---|---|
| Time (Hours) | (n = 3) |
| 2 | 29 |
| 4 | 59 |
| 6 | 77 |
| 8 | 86 |
| 12 | 89 |
| 16 | 89 |
| 20 | 88 |

### EXAMPLE 4

A controlled release tablet containing 10 mg of glipizide was prepared as in Example 1, having 25% Polyox N 750 (MW=300,000) and 51.76% Anhydrous Lactose.

The resulting tablet is dissolution tested in a pH 7.5 medium according to the procedure described in United States Pharmacopeia XXIII, Apparatus 2 @ 50 rpm and found to have the following release profile set forth in Table 4:

TABLE 4

| | Mean % Dissolved (pH 7.5) |
|---|---|
| Time (Hours) | (n = 3) |
| 2 | 25 |
| 4 | 55 |
| 6 | 77 |
| 8 | 88 |
| 12 | 93 |
| 16 | 94 |
| 20 | 93 |

The in-vitro release profile in pH 7.5 of the sustained release product prepared in Examples 2 to 5 is shown in FIG. 2.

### EXAMPLE 5

A controlled release tablet containing 10 mg of glipizide and having the following formula was prepared as in Example 1:

| | Percentage of tablet |
|---|---|
| I. Core | |
| Glipizide | 3.11% |
| Disintegrant (Explotab ®) | 9.33% |
| Anhydrous Lactose | 57.08% |
| Polyox N 60 K (MW = 2,000,000) | 18.66% |
| Magnesium Stearate | 0.93% |
| Colloidal Silicon Dioxide (e.g., Cab-O-Sil) | 0.47% |
| Glyceryl Monostearate | 3.73% |
| II. Seal Coating | |
| Opadry Clear | 2.92% |
| Sodium Chloride | 0.97% |
| III. Membrane | |
| Cellulose Acetate | 1.42% |
| Eudragit S100 | 0.71% |
| Triacetin | 0.27% |
| PEG 400 | 0.40% |

Table 5 provides mean plasma pharmacokinetic values based on non-fasting dosing with Example 5 and an equivalent dose of a reference standard (Glucotrol XL).

Table 5A provides mean plasma pharmacokinetic values based on fasting dosing with Example 5 and an equivalent dose of a reference standard (Glucotrol XL).

TABLE 5
Summany of Statistical Analysis N = 8
Log-Transformed Data

| | | | | | | | Mean | | Interval | |
|---|---|---|---|---|---|---|---|---|---|---|
| PK | | Least Squares Mean | | Geometric Mean | | | Square | Standard | (Lower Limit, | Power of |
| Variable | 90% Confidence | Ex. 5 | Reference | Ex. 5 | Reference | % Ratio | Error | Error | Upper Limit) | ANOVA |
| Cₘₐₓ | | 5.251 | 5.530 | 190.76 | 252.14 | 75.66 | 0.0735 | 0.1355 | (58.2, 98.5) | 0.2846 |
| AU₀₋ₜ | | 8.151 | 8.254 | 3467 | 3843 | 90.22 | 0.0613 | 0.1238 | (70.9, 115) | 0.3301 |
| AUC_{inf} | | 9.240 | 8.540 | 10301 | 5115 | 201.39 | 0.2809 | 0.2650 | (115, 355) | 0.1010 |

Non-Transformed Data

| | | | | | | Mean | | Interval | |
|---|---|---|---|---|---|---|---|---|---|
| PK | | Least Squares Mean | | | | Square | Standard | (Lower Limit, | Power of |
| Variable | 90% Confidence | Ex. 5 | Reference | Difference | % Ratio | Error | Error | Upper Limit) | ANOVA |
| Cₘₐₓ | | 200.28 | 255.16 | −54.88 | 78.49 | 3400.4576 | 29.1567 | (56.3, 101) | 0.3145 |
| Tₘₐₓ | | 12.00 | 8.50 | 3.50 | 141.18 | 3.1667 | 0.8898 | (121, 162) | 0.3630 |
| AUC₀₋ₜ | | 3646 | 3915 | −269.00 | 93.13 | 719482.7917 | 424.1117 | (72.1, 114) | 0.3433 |
| AUC_{inf} | | 12807 | 5451 | 7356.00 | 234.95 | 39203457.6875 | 3130.6332 | (113, 357) | 0.0586 |
| k_{clim} | | 0.0435 | 0.0579 | −0.0144 | 75.13 | 0.0005 | 0.0111 | (34.3, 116) | 0.1288 |
| t_{1/2} | | 43.56 | 13.56 | 30.00 | 321.24 | 736.7796 | 13.5718 | (108, 535) | 0.0528 |

Geometric means are based on least squares means of log transformed values.
Sample Size Calculations:
Log Transformed Cmax
Power 80%
%CV 27.62%
Ratio 0.76
N = Approximately 248

TABLE 5A
Summary of Statistical Analysis N = 7
Log-Transformed Data

| | | | | | | | | 90% Confidence | |
|---|---|---|---|---|---|---|---|---|---|
| | | | | | | Mean | | Interval | |
| PK | Least Squares Mean | | Geometric Mean | | | Square | Standard | (Lower limit, | Power of |
| Variable | Ex. 5 | Reference | Ex. 5 | Reference | % Ratio | Error | Error | Upper Limit) | ANOVA |
| Cₘₐₓ | 5.211 | 5.146 | 183.28 | 171.74 | 106.72 | 0.0323 | 0.0971 | (87.8, 130) | 0.4597 |
| AU₀₋ₜ | 8.003 | 7.906 | 2990 | 2714 | 110.17 | 0.0210 | 0.0783 | (94.1, 129) | 0.6288 |
| AUC_{inf} | 8.099 | 8.113 | 3291 | 3338 | 98.59 | 0.0297 | 0.0931 | (81.7, 119) | 0.4905 |

Non-Transformed Data

| | | | | | | | 90% Confidence | |
|---|---|---|---|---|---|---|---|---|
| | | | | | Mean | | Interval | |
| PK | Least Squares Mean | | | | Square | Standard | (Lower Limit, | Power of |
| Variable | Ex. 5 | Reference | Difference | % Ratio | Error | Error | Upper Limit) | ANOVA |
| Cₘₐₓ | 187.73 | 178.87 | 8.86 | 104.95 | 984.5981 | 16.9462 | (85.9, 124) | 0.4017 |
| Tₘₐₓ | 11.48 | 8.81 | 2.67 | 130.31 | 9.0667 | 1.6262 | (93.1, 167) | 0.1441 |
| AUC₀₋ₜ | 3279 | 3136 | 143.00 | 104.56 | 171342.1667 | 223.5504 | (90.2, 119) | 0.6161 |
| AUC_{inf} | 3595 | 3806 | −211.00 | 94.46 | 292818.6667 | 292.2421 | (79.0, 110) | 0.5551 |
| k_{clim} | 0.1104 | 0.0955 | 0.0149 | 115.60 | 0.0002 | 0.0066 | (102, 130) | 0.6397 |
| t_{1/2} | 7.74 | 9.78 | −2.04 | 79.14 | 8.6854 | 1.5916 | (46.4, 112) | 0.1716 |

Geometric means are based on least squares means of log transformed values.
Sample Size Calculations:
Log Transformed Cmax
Power 80%
%CV 18.12%
Ratio 1.07
N = Approximately 26

### EXAMPLE 6

A controlled release tablet containing 10 mg of glipizide and having the following formula is prepared as in Example 1:

| | Percentage of tablet |
|---|---|
| I. Core | |
| Glipizide | 2.91% |
| Disintegrant (Explotab ®) | 8.75% |
| Anhydrous Lactose | 53.52% |
| Polyox N 60 K (MW = 2,000,000) | 17.50% |
| Magnesium Stearate | 0.88% |
| Colloidal Silicon Dioxide (e.g., Cab-O-Sil) | 0.44% |
| Glyceryl Monostearate | 3.50% |
| II. Seal Coating | |
| Opadry Clear | 5.09% |
| Sodium Chloride | 1.67% |
| III. Membrane | |
| Cellulose Acetate | 1.270% |
| Eudragit S100 | 0.635% |
| Triacetin | 0.238% |
| PEG 400 | 0.357% |
| IV. Color Coating | |
| Opadry White | 3.00% |
| Candelilla Wax Powder | 0.03% |

Table 6 provides mean plasma pharmacokinetic values based on fasting dosing with Example 6 and an equivalent dose of a reference standard (Glucotrol XL).

TABLE 6

| | Ex.6 | | Ref. | | G-Mean | A-Mean |
|---|---|---|---|---|---|---|
| | Mean | % CV | Mean | % CV | ratio | Ratio |
| Cmax | 234.19 | 32.51 | 188.93 | 15.84 | 1.193 | 1.244 |
| AUC 0˜t | 3501.87 | 25.76 | 3397.46 | 24.76 | 1.030 | 1.045 |
| AUC | 3789.53 | 22.56 | 3712.56 | 23.26 | 1.024 | 1.037 |
| 0˜inf | | | | | | |
| Tmax | 9.13 | 42.01 | 6.50 | 45.04 | 1.407 | 1.500 |

### EXAMPLE 7

A controlled release tablet containing 10 mg of glipizide and having the following formula is prepared as in Example 1:

| | Percentage of |
|---|---|
| | tablet |
| I. Core | |
| Glipizide | 3.01% |
| Disintegrant (Explotab ®) | 9.05% |
| Anhydrous Lactose | 55.35% |
| Polyox N 60 K (MW = 2,000,000) | 18.10% |
| Magnesium Stearate | 0.90% |
| Colloidal Silicon Dioxide (e.g., Cab-O-Sil) | 0.45% |
| Glyceryl Monostearate | 3.62% |
| II. Seal Coating | |
| Opadry Clear | 2.83% |
| Sodium Chloride | 0.94% |
| III. Membrane | |
| Cellulose Acetate | 1.38% |
| Eudragit S100 | 0.69% |
| Triacetin | 0.26% |
| PEG 400 | 0.39% |
| IV. Color Coating | |
| Opadry White | 3.00% |
| Candelilla Wax Powder | 0.03% |

Table 7 provides mean plasma pharmacokinetic values based on fasting dosing with Example 7 and an equivalent dose of a reference standard (Glucotrol XL).

Table 7A provides individual plasma concentration (ng/ml) following dosing with Example 7.

TABLE 7
Summary of Statistical Analysis N = 9
Log-Transformed Data

| | | | | | | | | 90% Confidence | |
|---|---|---|---|---|---|---|---|---|---|
| | | | | | | Mean | | Interval | |
| PK | Least Squares Mean | | Geometric Mean | | | Square | Standard | (Lower limit, | Power of |
| Variable | Ex. 5 | Reference | Ex. 5 | Reference | % Ratio | Error | Error | Upper Limit) | ANOVA |
| Cₘₐₓ | 5.481 | 5.029 | 240.09 | 152.78 | 157.15 | 0.1265 | 0.1186 | (128, 194) | 0.2992 |
| AU₀₋ₜ | 8.144 | 8.016 | 3443 | 3029 | 113.67 | 0.0611 | 0.0824 | (98.3, 131) | 0.5394 |
| AUC_{inf} | 8.304 | 8.185 | 4040 | 3587 | 112.63 | 0.0389 | 0.0658 | (100, 126) | 0.7314 |

Non-Transformed Data

| | | | | | | | 90% Confidence | |
|---|---|---|---|---|---|---|---|---|
| | | | | | Mean | | Interval | |
| PK | Least Squares Mean | | | | Square | Standard | (Lower Limit, | Power of |
| Variable | Ex. 5 | Reference | Difference | % Ratio | Error | Error | Upper Limit) | ANOVA |
| Cₘₐₓ | 273.49 | 160.72 | 112.77 | 170.17 | 10619.4578 | 34.3503 | (133, 208) | 0.1099 |
| Tₘₐₓ | 8.33 | 11.67 | −3.34 | 71.38 | 14.0668 | 1.2503 | (52.6, 90.3) | 0.2950 |
| AUC₀₋ₜ | 3716 | 3287 | 429.00 | 113.05 | 483607.5767 | 231.8063 | (101, 125) | 0.5774 |
| AUC_{inf} | 4271 | 3827 | 444.00 | 111.60 | 565083.2381 | 250.5734 | (100, 123) | 0.6410 |
| kₑₗᵢₘ | 0.0869 | 0.0887 | −0.0018 | 97.97 | 0.0011 | 0.0109 | (76.4, 120) | 0.2365 |
| t_{1/2} | 9.53 | 10.39 | −0.86 | 91.72 | 26.7951 | 1.7255 | (62.5, 121) | 0.1505 |

Geometric means are based on least squares means of log transformed values.

TABLE 7A

| | 0.00 | 2.00 | 3.00 | 4.00 | 5.00 | 6.00 | 8.00 | 10.00 | 12.00 |
|---|---|---|---|---|---|---|---|---|---|
| Subject | hr. | hr. | hr. | hr. | hr | hr. | hr. | hr. | hr. |
| 1 | 0.00 | 0.00 | 0.00 | 36.45 | 125.90 | 148.20 | 172.54 | 176.49 | 178.95 |
| 2 | 0.00 | 0.00 | 32.26 | 95.39 | 152.37 | 168.11 | 156.93 | 174.97 | 168.37 |
| 3 | 0.00 | 0.00 | 27.39 | 101.61 | 143.86 | 140.67 | 123.68 | 95.48 | 65.56 |
| 4 | 0.00 | 11.57 | 70.43 | 112.60 | 104.40 | 106.99 | 156.00 | 173.06 | 173.85 |
| 5 | 0.00 | 0.00 | 0.00 | 33.21 | 119.36 | 121.68 | 78.77 | 65.41 | 59.76 |
| 6 | 0.00 | 0.00 | 15.05 | 65.09 | 121.49 | 112.75 | 86.12 | 103.67 | 124.41 |
| 7 | 0.00 | 0.00 | 37.97 | 38.61 | 32.28 | 36.71 | 46.25 | 79.55 | 86.20 |
| 8 | 0.00 | 0.00 | 24.69 | 89.01 | 106.96 | 93.14 | 81.73 | 96.88 | 99.47 |
| 9 | 0.00 | 0.00 | 47.30 | 110.28 | 149.61 | 216.61 | 159.50 | 140.98 | 135.95 |
| N | 9 | 9 | 9 | 9 | 9 | 9 | 9 | 9 | 9 |
| Mean | 0.00 | 1.29 | 28.34 | 75.81 | 117.36 | 127.20 | 117.95 | 122.94 | 121.39 |
| STDEV | 0.00 | 3.86 | 22.46 | 32.84 | 36.36 | 50.36 | 45.70 | 43.89 | 46.27 |
| %CV | | 300.00 | 79.25 | 43.32 | 30.98 | 39.59 | 38.75 | 35.70 | 38.12 |
| MIN | 0.00 | 0.00 | 0.00 | 33.21 | 32.28 | 36.71 | 46.25 | 65.41 | 59.76 |
| MAX | 0.00 | 11.57 | 70.43 | 112.60 | 152.37 | 216.61 | 172.54 | 176.49 | 178.95 |

| | 16.00 | 20.00 | 24.00 | 30.00 | 36.00 | 48.00 | 60.00 | 72.00 |
|---|---|---|---|---|---|---|---|---|
| Subject | hr. | hr. | hr. | hr. | hr. | hr. | hr. | hr. |
| 1 | 188.75 | 132.60 | 100.36 | 41.48 | 20.88 | 0.00 | 0.00 | 0.00 |
| 2 | 115.65 | 93.46 | 65.47 | 42.91 | 17.74 | 0.00 | 0.00 | 0.00 |
| 3 | 31.67 | 18.30 | 11.40 | 0.00 | 0.00 | 0.00 | 0.00 | 0.00 |
| 4 | 117.22 | 74.76 | 59.34 | 31.45 | 14.36 | 0.00 | 0.00 | 0.00 |
| 5 | 51.00 | 39.29 | 38.68 | 63.20 | 76.38 | 20.19 | 20.95 | 0.00 |
| 6 | 124.35 | 108.28 | 97.37 | 66.12 | 34.33 | 0.00 | 0.00 | 0.00 |
| 7 | 66.97 | 52.99 | 43.76 | 10.80 | 0.00 | 0.00 | 0.00 | 0.00 |
| 8 | 74.74 | 76.06 | 65.21 | 23.48 | 0.00 | 0.00 | 0.00 | 0.00 |
| 9 | 113.38 | 82.43 | 70.50 | 49.30 | 24.97 | 0.00 | 0.00 | 0.00 |
| N | 9 | 9 | 9 | 9 | 9 | 9 | 9 | 9 |
| Mean | 98.19 | 75.35 | 61.34 | 36.53 | 20.96 | 2.24 | 2.33 | 0.00 |
| STDEV | 47.38 | 34.99 | 27.97 | 22.38 | 24.08 | 6.73 | 6.98 | 0.00 |
| %CV | 48.25 | 46.44 | 45.59 | 61.28 | 114.87 | 300.00 | 300.00 | |
| MIN | 31.67 | 18.30 | 11.40 | 0.00 | 0.00 | 0.00 | 0.00 | 0.00 |
| MAX | 188.75 | 132.60 | 100.36 | 66.12 | 76.38 | 20.19 | 20.95 | 0.00 |

Table 7B provides mean plasma pharmacokinetic values based on fasting dosing with Example 7 and an equivalent dose of a reference standard (Glucotrol XL).

TABLE 7B

| | Ex.7 | | Ref. | | G-Mean | A-Mean |
|---|---|---|---|---|---|---|
| | Mean | % CV | Mean | % CV | ratio | Ratio |
| Cmax | 148.58 | 28.68 | 180.25 | 25.49 | 0.815 | 0.845 |
| AUC 0˜72 | 2723.48 | 32.25 | 2866.40 | 33.89 | 0.952 | 0.991 |
| Tmax | 9.33 | 42.52 | 8.22 | 38.83 | 1.125 | 1.206 |

reference standard (Glucotrol XL).

TABLE 7C

| | Ex.7 | | Ref. | | G-Mean | A-Mean |
|---|---|---|---|---|---|---|
| | Mean | % CV | Mean | % CV | ratio | Ratio |
| Cmax | 235.74 | 19.91 | 246.99 | 29.59 | 0.976 | 1.034 |
| AUC 0˜72 | 3163.58 | 27.00 | 3362.33 | 30.64 | 0.947 | 0.964 |
| Tmax | 8.11 | 49.49 | 9.56 | 25.16 | 0.810 | 0.906 |

### EXAMPLE 8

A controlled release tablet containing 10 mg of glipizide and having the following formula is prepared as in Example 1:

| | Percentage of |
|---|---|
| | tablet |
| I. Core | |
| Glipizide | 3.11% |
| Disintegrant (Explotab ®) | 9.33% |
| Anhydrous Lactose | 57.08% |
| Polyox N 60 K (MW = 2,000,000) | 18.66% |
| Magnesium Stearate | 0.93% |
| Colloidal Silicon Dioxide (e.g., Cab-O-Sil) | 0.47% |
| Glyceryl Monostearate | 3.73% |
| II. Seal Coating | |
| Opadry Clear | 2.92% |
| Sodium Chloride | 0.97% |
| III. Membrane | |
| Cellulose Acetate | 1.76% |
| Eudragit S100 | 0.88% |
| Triacetin | 0.33% |
| PEG 400 | 0.50% |

Table 8 provides mean plasma pharmacokinetic values based on fasting dosing with Example 8 and an equivalent dose of a reference standard (Glucotrol XL).

TABLE 8

| | Ex.8 | | Ref. | | G-Mean | A-Mean |
|---|---|---|---|---|---|---|
| | Mean | % CV | Mean | % CV | ratio | Ratio |
| Cmax | 147.46 | 22.47 | 180.25 | 25.49 | 0.820 | 0.843 |
| AUC 0˜72 | 2664.54 | 34.51 | 2866.40 | 33.89 | 0.932 | 0.962 |
| Tmax | 10.33 | 33.52 | 8.22 | 38.83 | 1.280 | 1.456 |

Table 8A provides mean plasma pharmacokinetic values based on non-fasting dosing with Example 8 and an equivalent dose of a reference standard (Glucotrol XL).

TABLE 8A

| | Ex.8 | | Ref. | | G-Mean | A-Mean |
|---|---|---|---|---|---|---|
| | Mean | % CV | Mean | % CV | ratio | Ratio |
| Cmax | 192.04 | 20.84 | 246.99 | 29.59 | 0.792 | 0.832 |
| AUC 0˜72 | 2781.97 | 42.54 | 3362.33 | 30.64 | 0.798 | 0.821 |
| Tmax | 8.33 | 30.59 | 9.56 | 25.16 | 0.863 | 0.906 |

### EXAMPLE 9

A controlled release tablet containing 10 mg of glipizide and having the following formula is prepared as in Example 1:

| | Percentage of |
|---|---|
| | tablet |
| I. Core | |
| Glipizide | 2.94% |
| Disintegrant (Explotab ®) | 8.84% |
| Anhydrous Lactose | 54.04% |
| Polyox N 60 K (MW = 2,000,000) | 17.68% |
| Magnesium Stearate | 0.88% |
| Colloidal Silicon Dioxide (e.g., Cab-O-Sil) | 0.44% |
| Glyceryl Monostearate | 3.54% |
| Butylated Hydroxytoluene (BHT) | 0.03% |
| II. Seal Coating | |
| Opadry Clear | 3.86% |
| Sodium Chloride | 1.29% |
| III. Membrane | |
| Cellulose Acetate | 1.73% |
| Eudragit S100 | 0.86% |
| Triacetin | 0.32% |
| PEG 400 | 0.53% |
| IV. Color Coating | |
| Opadry White | 3.00% |
| Candelilla Wax Powder | 0.03% |

The resulting tablet is tested in a medium (pH 7.5) according to the procedure described in Unites States Pharmacopeia XXIII, Apparatus 2 @ 50 rpm. Table 9 provides mean plasma pharmacokinetic values based on fasting dosing with Example 9 and an equivalent dose of a reference standard (Glucotrol XL).

TABLE 9
Summary of Statistical Analysis N = 27

| | | | | 90% Confidence |
|---|---|---|---|---|
| | | | | Interval |
| | Geometeric Means | | | (Lower Upper Limit, |
| PK Parameter | Test | References | % Ratio | Limit) |

Ln-Transformed Data

| | | | | |
|---|---|---|---|---|
| Cₘₐₓ | 146.01 | 140.22 | 104.13 | (95.76, 113.23) |
| AUC₀₋ₜ | 2310.22 | 2583.88 | 89.41 | (81.71, 97.83) |
| AUC_{inf} | 2550.10 | 2784.78 | 91.57 | (83.88, 99.97) |

Non-Transformed Data

| | | | | |
|---|---|---|---|---|
| Cₘₐₓ | 153.42 | 147.26 | 104.18 | (95.34, 113.02) |
| AUC₀₋ₜ | 2580.93 | 2802.05 | 92.11 | (85, 99.22) |
| AUC_{inf} | 2833.29 | 3014.16 | 94.00 | (86.99, 101.01) |
| Tₘₐₓ | 9.27 | 9.49 | 97.71 | (83.97, 111.44) |
| Kₑₗᵢₘ | 0.118 | 0.1154 | 96.88 | (87.7, 111.44) |
| t_{1/2} | 7.51 | 7.12 | 105.50 | (94.86, 116.14) |

Geometric means are based on least squares means of in-transformed values. reference standard (Glucotrol XL).

TABLE 9A

| | Ex.9 | | Ref. | | G-Mean | A-Mean |
|---|---|---|---|---|---|---|
| | Mean | % CV | Mean | % CV | ratio | Ratio |
| Cmax | 181.16 | 44.78 | 202.52 | 53.01 | 0.900 | 0.934 |
| AUC 0˜t | 2253.77 | 51.01 | 3048.99 | 89.09 | 0.844 | 0.899 |
| AUC 0˜inf | 2704.80 | 39.09 | 3473.92 | 77.63 | 0.884 | 0.982 |
| Tmax | 8.86 | 28.73 | 9.43 | 41.92 | 0.960 | 0.995 |

reference standard (Glucotrol XL) in an N = 23 biostudy.

TABLE 9B
Summary of Statistical Analysis N = 23
Ln-Transformed Data

| | | | | | | | 90% Confidence | | |
|---|---|---|---|---|---|---|---|---|---|
| | | | | | | Mean | Interval | P-values for | |
| PK | Least Squares Mean | | Geometric Mean | | | Square | (Lower Limit, | Product | Power of |
| Variable | Test | Reference | Test | Reference | % Ration | Error | Upper Limit) | Effects | ANOVA |
| Cₘₐₓ | 4.989 | 5.122 | 146.80 | 167.67 | 87.55 | 0.02989 | (80.19, 95.59) | 0.0165 | 0.9863 |
| AUC₀₋ₜ | 7.700 | 7.728 | 2207.84 | 2270.31 | 97.25 | 0.04919 | (86.89, 108.84) | 0.6743 | 0.9013 |
| AUC _{inf} | 7.796 | 7.811 | 2430.80 | 2468.33 | 98.48 | 0.05752 | (87.19, 111.24) | 0.8308 | 0.8520 |

Non-Transformed Data

| | | | | | | P-values for | |
|---|---|---|---|---|---|---|---|
| PK | Least Squares Mean | | | | 90% Confidence Interval | Product | Power of |
| Variable | Test | Reference | % Ratio | Mean Square Error | (Lower Limit, Upper Limit) | Effects | ANOVA |
| Cₘₐₓ | 151.86 | 174.67 | 86.94 | 736.32 | (79.05, 94.83) | 0.0096 | 0.9859 |
| AUC₀₋ₜ | 2363.63 | 2389.72 | 98.91 | 296695.28 | (87.33, 110.49) | 0.8727 | 0.8091 |
| AUC_{inf} | 2586.77 | 2585.83 | 100.04 | 361313.08 | (88.23, 111.84) | 0.9958 | 0.7937 |
| Tₘₐₓ | 9.23 | 7.42 | 124.34 | 5.5904 | (108.16, 140.52) | 0.0171 | 0.5277 |
| Kₑₗᵢₘ | 0.1021 | 0.1152 | 88.61 | 0.00290 | (64.85, 112.37) | 0.4188 | 0.2822 |
| t_{1/2} | 8.33 | 7.45 | 111.83 | 11.83308 | (88.37, 135.29) | 0.3955 | 0.2881 |

Geometric means are based on least squares means of Ln-transformed values.

The resulting tablet is dissolution tested in a pH 7.5 medium according to the procedure described in United States Pharmacopeia XXIII, Apparatus 2 @ 50 rpm, and was found to have the following mean release profile set forth in Table 9C.

TABLE 9C

| Time (Hours) | % Dissolved (pH 7.5) |
|---|---|
| 2 | 11 |
| 6 | 40 |
| 12 | 94 |
| 16 | 108 |

TABLE 9D provides the individual dissolution profiles of the above dissolution test of samples V1 through V12 using Example 9 as compared to the individual and mean results of GLUCOTROL XL 10MG.

TABLE 9D

| | | | | | | | | | | | | | | | |
|---|---|---|---|---|---|---|---|---|---|---|---|---|---|---|---|
| Time (hr) | V1 | V2 | V3 | V4 | V5 | V6 | V7 | V8 | V9 | V10 | V11 | V12 | range | Avg | % RSO |

Example 9 Tablet Dissolution Profile

| | | | | | | | | | | | | | | | | |
|---|---|---|---|---|---|---|---|---|---|---|---|---|---|---|---|---|
| 0 | 0 | 0 | 0 | 0 | 0 | 0 | 0 | 0 | 0 | 0 | 0 | 0 | 0 | 0 | 0 | 115.8 |
| 1 | 0 | 0 | 0 | 0 | 0 | 0 | 0 | 0 | 0 | 0 | 1 | 0 | 0 | 1 | 0 | 245.3 |
| 2 | 18 | 10 | 4 | 3 | 10 | 8 | 10 | 13 | 14 | 7 | 12 | 3 | 3 | 15 | 9 | 44.4 |
| 4 | 29 | 28 | 21 | 18 | 22 | 25 | 23 | 29 | 28 | 24 | 28 | 16 | 18 | 29 | 24 | 18.6 |
| 6 | 43 | 46 | 37 | 35 | 35 | 42 | 38 | 44 | 43 | 41 | 42 | 31 | 31 | 46 | 40 | 11.8 |
| 8 | 58 | 60 | 52 | 50 | 62 | 61 | 54 | 58 | 58 | 58 | 57 | 52 | 50 | 61 | 58 | 6.8 |
| 12 | 88 | 98 | 94 | 91 | 92 | 103 | 95 | 95 | 101 | 86 | 88 | 90 | 88 | 103 | 94 | 4.9 |
| 16 | 104 | 109 | 106 | 106 | 105 | 110 | 107 | 103 | 111 | 105 | 100 | 111 | 100 | 111 | 106 | 3.2 |
| 20 | 108 | 112 | 110 | 106 | 105 | 110 | 108 | 106 | 112 | 108 | 104 | 110 | 104 | 112 | 108 | 2.6 |

Glucotrol XL 10 Reference Tablet Dissolution Profile

| | | | | | | | | | | | | | | | | |
|---|---|---|---|---|---|---|---|---|---|---|---|---|---|---|---|---|
| 0 | 0 | 0 | 0 | 0 | 0 | 0 | 0 | 0 | 0 | 0 | 0 | 0 | 0 | 0 | 0 | −904.5 |
| 1 | 1 | 1 | 1 | 1 | 1 | 1 | 1 | 1 | 1 | 1 | 1 | 1 | 1 | 1 | 1 | 25.1 |
| 2 | 3 | 5 | 6 | 3 | 1 | 5 | 1 | 2 | 1 | 0 | 4 | 2 | 0 | 6 | 3 | 69.6 |
| 4 | 16 | 20 | 27 | 18 | 13 | 23 | 13 | 20 | 17 | 13 | 25 | 20 | 13 | 27 | 19 | 24.8 |
| 6 | 33 | 36 | 45 | 37 | 26 | 40 | 28 | 38 | 35 | 28 | 41 | 37 | 26 | 45 | 38 | 16.2 |
| 8 | 50 | 54 | 54 | 55 | 41 | 59 | 44 | 56 | 54 | 44 | 61 | 66 | 41 | 64 | 53 | 13.3 |
| 12 | 82 | 89 | 99 | 90 | 79 | 85 | 76 | 83 | 80 | 76 | 97 | 90 | 75 | 98 | 88 | 8.2 |
| 16 | 110 | 112 | 112 | 112 | 107 | 110 | 101 | 111 | 114 | 103 | 113 | 100 | 101 | 114 | 109 | 3.6 |
| 20 | 112 | 113 | 113 | 113 | 112 | 111 | 112 | 114 | 116 | 110 | 118 | 112 | 110 | 116 | 113 | 1.5 |

The resulting tablet was also tested in a medium (pH 6.5) according to the procedure described in United States Pharmacopeia XXIII, Apparatus 2 @ 50 rpm and found to have the following release profile set forth in Table 9E.

TABLE 9E

| Time (Hours) | % Dissolved (pH 6.5) |
|---|---|
| 2 | 16 |
| 6 | 37 |
| 12 | 74 |
| 16 | 93 |

TABLE 9F provides the individual dissolution profiles of the above dissolution test of samples V1 through V12 using Example 9 as compared to the individual and mean results of GLUCOTROL XL 10MG.

TABLE 9F

| | | | | | | | | | | | | | | | |
|---|---|---|---|---|---|---|---|---|---|---|---|---|---|---|---|
| Time (hr) | V1 | V2 | V3 | V4 | V5 | V6 | V7 | V8 | V9 | V10 | V11 | V12 | range | Avg | % RSO |

Example 9 Tablet Dissolution Profile

| | | | | | | | | | | | | | | | | |
|---|---|---|---|---|---|---|---|---|---|---|---|---|---|---|---|---|
| 0 | 0 | 0 | 0 | 0 | 0 | 0 | 0 | 0 | 0 | 0 | 0 | 0 | 0 | 0 | 0 | −723.5 |
| 1 | 12 | 18 | 13 | 15 | 9 | 10 | 13 | 16 | 10 | 16 | 8 | 14 | 8 | 18 | 13 | 23.9 |
| 2 | 16 | 22 | 22 | 22 | 12 | 12 | 13 | 16 | 11 | 19 | 13 | 17 | 11 | 22 | 16 | 26.3 |
| 4 | 22 | 47 | 43 | 56 | 21 | 10 | 16 | 21 | 18 | 37 | 30 | 26 | 18 | 47 | 28 | 36.6 |
| 6 | 32 | 65 | 49 | 49 | 26 | 28 | 26 | 34 | 25 | 46 | 37 | 36 | 25 | 88 | 37 | 28.7 |
| 8 | 45 | 86 | 62 | 61 | 40 | 48 | 38 | 52 | 38 | 57 | 46 | 40 | 35 | 65 | 49 | 20.6 |
| 12 | 71 | 87 | 94 | 79 | 55 | 62 | 81 | 85 | 61 | 80 | 54 | 67 | 58 | 94 | 74 | 16.4 |
| 16 | 91 | 96 | 99 | 98 | 84 | 81 | 105 | 102 | 84 | 95 | 88 | 88 | 81 | 108 | 99 | 8.3 |
| 20 | 98 | 88 | 103 | 100 | 85 | 84 | 112 | 100 | 88 | 101 | 88 | 101 | 84 | 112 | 100 | 4.5 |

Glucotrol XL 10 Reference Tablet Dissolution Profile

| | | | | | | | | | | | | | | | | |
|---|---|---|---|---|---|---|---|---|---|---|---|---|---|---|---|---|
| 0 | 0 | 0 | 0 | 0 | 0 | 0 | 0 | 0 | 0 | 0 | 0 | 0 | 0 | 0 | 0 | 106.8 |
| 1 | 0 | 0 | 0 | 4 | 0 | 4 | 1 | 1 | 1 | 1 | 1 | 1 | 0 | 4 | 1 | 145.8 |
| 2 | 0 | 2 | 1 | 4 | 3 | 5 | 2 | 4 | 1 | 4 | 1 | 7 | 0 | 7 | 3 | 88.3 |
| 4 | 11 | 19 | 18 | 20 | 20 | 19 | 17 | 21 | 8 | 10 | 18 | 26 | 8 | 26 | 18 | 26.2 |
| 6 | 25 | 36 | 33 | 38 | 38 | 34 | 33 | 37 | 21 | 32 | 33 | 44 | 21 | 44 | 34 | 17.3 |
| 8 | 43 | 63 | 61 | 68 | 68 | 50 | 51 | 58 | 35 | 48 | 51 | 63 | 36 | 63 | 61 | 14.0 |
| 12 | 77 | 84 | 85 | 90 | 87 | 81 | 83 | 91 | 88 | 79 | 83 | 98 | 66 | 98 | 84 | 9.4 |
| 16 | 103 | 111 | 110 | 111 | 111 | 107 | 107 | 112 | 92 | 105 | 108 | 114 | 92 | 114 | 108 | 5.5 |
| 20 | 112 | 114 | 114 | 114 | 115 | 114 | 111 | 115 | 106 | 111 | 114 | 118 | 106 | 116 | 113 | 2.2 |

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

What is claimed is:

1. A controlled release oral dosage form comprising:
(a) a core comprising:
(i) a sulfonylurea or a pharmaceutically acceptable salt thereof;
(ii) a pharmaceutically acceptable polymer;
(b) a membrane surrounding the core which is permeable to the sulfonylurea and gastrointestinal fluid; said dosage form being suitable for providing once-a-day oral administration of the sulfonylurea.

2. The controlled release oral dosage form of claim 1, wherein said sulfonylurea is glipizide or a pharmaceutically acceptable salt thereof.

3. The controlled release oral dosage form of claim 1, wherein said core further includes a binding agent.

4. The controlled release oral dosage form of claim 1, wherein said core further includes an absorption enhancer.

5. The controlled release oral dosage form of claim 1, wherein said sulfonylurea and said polymer are at least partially interdispersed.

6. The controlled release oral dosage form of claim 5, wherein said sulfonylurea and said polymer are uniformly dispersed.

7. The controlled release oral dosage form of claim 1, wherein said membrane comprises a plasticizer.

8. The controlled release oral dosage form of claim 1, wherein said membrane comprises triacetin, polyethylene glycol or mixtures thereof.

9. The controlled release oral dosage form of claim 1, wherein said pharmaceutically acceptable polymer is polyethylene oxide.

10. The controlled release oral dosage form of claim 2, which exhibits the following dissolution profiles when tested in a USP type 2 apparatus at 50 rpm in 900 ml of medium (pH 7.5 phosphate buffer) and at 37 C:
from 0 to about 40% of glipizide or salt thereof is released after 2 hours;
from about 20% to about 90% of glipizide or salt thereof released after 6 hours;
not less than about 60% of glipizide or salt thereof released after 12 hours;
not less than about 70% of glipizide or salt thereof released after 16 hours;
and not less than about 80% of glipizide or salt thereof released after 20 hours.

11. The controlled release oral dosage form of claim 2, which exhibits the following dissolution profiles when tested in a USP type 2 apparatus at 50 rpm in 900 ml of medium (pH 6.5 phosphate buffer) and at 37 C:
from 0 to about 25% of glipizide or salt thereof released after 2 hours;
from about 10% to about 55% of glipizide or salt thereof released after 6 hours;
from about 40% to about 95% of glipizide or salt thereof released after 12 hours;
and not less than about 70% of glipizide or salt thereof released after 16 hours.

12. The controlled release oral dosage form of claim 2, which after oral administration of a single dose to a human patient, provides a mean plasma concentrations of glipizide of from about 10 to about 150 ng/ml at 4 hours after administration, from about 75 to about 350 ng/ml at 8 hours after administration; from about 65 to about 275 ng/ml at 12 hours after administration and from about 25 to about 125 ng/ml at 24 hours after administration, based on a 10 mg dose of glipizide.

13. The controlled release oral dosage form of claim 12, which after oral administration of a single dose to a human patient, provides a mean plasma concentrations of glipizide of from about 15 to about 100 ng/ml at 4 hours after administration, from about 75 to about 150 ng/ml at 8 hours after administration; from about 100 to about 180 ng/ml at 12 hours after administration and from about 30 to about 100 ng/ml at 24 hours after administration, based on a 10 mg dose of glipizide.

14. The controlled release oral dosage form of claim 2, which after oral administration of a single dose to a human patient, provides a mean plasma concentrations of glipizide of from about 5 to about 75 ng/ml at 4 hours after administration, from about 35 to about 175 ng/ml at 8 hours after administration; from about 30 to about 135 ng/ml at 12 hours after administration and from about 10 to about 65 ng/ml at 24 hours after administration, based on a 5 mg dose of glipizide.

15. The controlled release oral dosage form of claim 14, which after oral administration of a single dose to a human patient, provides a mean plasma concentrations of glipizide of from about 7 to about 50 ng/ml at 4 hours after administration, from about 35 to about 75 ng/ml at 8 hours after administration; from about 50 to about 100 ng/ml at 12 hours after administration and from about 15 to about 50 ng/ml at 24 hours after administration, based on a 5 mg dose of glipizide.

16. The controlled release solid oral dosage form of claim 1 wherein said membrane further includes at least one passageway in the membrane.

17. A method for lowering blood glucose levels in human patients needing treatment for non-insulin-dependent diabetes mellitus (NIDDM), comprising orally administering to human patients on a once-a-day basis a dose of controlled release dosage form comprising:
(a) a core comprising:
(i) a sulfonylurea or a pharmaceutically acceptable salt thereof;
(ii) a pharmaceutically acceptable polymer;
(b) a membrane surrounding the core which is permeable to the sulfonylurea and gastrointestinal fluid; said dosage form being suitable for providing once-a-day oral administration of the sulfonylurea.

18. The method of claim 17, wherein said sulfonylurea is glipizide.

19. The method of claim 18, in which the once-a-day dose of the glipizide is administered in the morning prior to breakfast.

20. The method of claim 14, in which the once-a-day dose of glipizide is administered at fasted state.

21. The controlled release dosage form of claim 1, wherein said dosage form provides a mean time to maximum plasma concentration (Tₘₐₓ) of said sulfonylurea at from about 4 to about 16 hours after oral administration.

22. The controlled release dosage form of claim 1, wherein said dosage form provides a mean time to maximum plasma concentration (Tₘₐₓ) of said sulfonylurea at from about 6 to about 12 hours after oral administration.

23. The dosage form of claim 1 wherein said membrane comprises less than about 10% of the total weight of the dosage form.

24. The dosage form of claim 1 wherein said polymer has a molecular weight greater than about 350,000 and less than about 4,000,000.

25. The method of claim 1 wherein said polymer of said dosage form is polyethylene oxide.

26. The dosage form of claim 1 wherein said membrane is substantially free of sodium chloride.

27. A controlled release oral dosage form comprising:
(a) a core comprising:
(i) an agent consisting essentially of a sulfonylurea or a pharmaceutically acceptable salt thereof;
(ii) a pharmaceutically acceptable polymer; and
(b) a membrane surrounding the core which is permeable to the sulfonylurea and gastrointestinal fluid; said dosage form being suitable for providing once-a-day oral administration of the sulfonylurea.

28. The controlled release oral dosage form of claim 27 wherein said core further comprises a disintegrant.

29. The controlled release oral dosage form of claim 28 wherein said disintegrant is sodium starch glycolate.

30. The controlled release oral dosage form of claim 1 further comprising a disintegrant.

31. The controlled release oral dosage form of claim 28 wherein said disintegrant is sodium starch glycolate.
